(19) United States
(12) Patent Application Publication
(10) Pub. No.: US 2001/0053521 A1
(43) Pub. Date: Dec. 20, 2001

(54) Amplification of analyte detection by substrates having particle structures with receptors

(76) Inventors: David I. Kreimer, Berkeley, CA (US); Oleg A. Yevin, Walnut Creek, CA (US); Thomas H. Nufert, Walnut Creek, CA (US)

Correspondence Address: Sheldon R. Meyer, FLIESLER DUBB MEYER & LOVEJOY LLP, Suite 400, Four Embarcadero Center, San Francisco, CA, 94111-4156, US

(21) Appl. No.: 09/815,909

(22) Filed: Mar. 23, 2001

Related U.S. Application Data
(63) Continuation-in-part of application No. 09/670,453, filed on Sep. 26, 2000.
(60) Provisional application No. 60/156,195, filed on Sep. 27, 1999.

(51) Int. Cl. C12Q 1/68; G01N 33/53; G01N 33/542; G01J 3/44

(52) U.S. Cl. 435/6; 438/1; 356/301; 435/7.9

(57) ABSTRACT

This invention comprises devices, compositions and methods for detecting analytes in complex solutions. In certain embodiments of this invention, the receptor molecules can be lacking in a Raman signal generating moiety that is present in an analyte molecule, so that binding of the analyte to the receptor provides the array with the Raman signal generating moiety. In other embodiments, binding of a receptor to an analyte is detected by observing an alteration or shift of Raman spectral features upon forming of an analyte-receptor associate. Enzymes can be detected by binding to receptors comprising materials that bind to the active site of the enzyme. Antibodies can be detected by formation of an antibody-antigen complex, wherein the antigen is part of a receptor. Analytes that can be detected using these methods include nucleic acids, proteins, and other molecules that can specifically bind to the arrays. Particle structures are disclosed that enhance the Raman a signal produced by an analyte through surface and resonance phenomena. Novel methods are presented for manufacturing particle structures and for attaching receptor molecules to areas of enhanced resonance on the particle structures, thereby producing Raman signals that can be used for highly specific, sensitive assays of biological and other molecules of interest.

### RELATED APPLICATIONS

This application is a continuation-in-part of U.S. application, Ser. No. 09/670,453, filed Sep. 26 2000, which claimed priority under 35 U.S.C. §119 to U.S. Provisional Patent Application Serial No. 60/156,195, now abandoned. This application is related to U.S. patent application Ser. No. 09/669,369, filed Sep. 26, 2000, U.S. patent application Ser. No. 09/669,796, filed Sep. 26, 2000, and U.S. Patent Application titled “Surfaces Having Particle Structures with Broad Range Radiation Absorptivity”, inventors Oleg A. Yevin, Thomas H. Nufert and David I. Kreimer, filed Mar. 23, 2001. Each of these Patent Applications is herein incorporated fully by reference.

### BACKGROUND OF THE INVENTION

1. Field of the Invention

This invention relates to the manufacture of particle structures for analyte detection. Specifically, the invention relates to the manufacture of particle structures having receptor molecules attached to resonance domains within the particle structures. More specifically, the invention relates to the use of particle structures having receptor molecules for the detection of analytes using Raman spectroscopy.

2. Description of Related Art

The detection and quantification of molecules or “analytes” in complex mixtures containing small amounts of analyte and large numbers and amounts of other materials is a continuing challenge. As more interest is focused upon the roles of biological molecules in physiology and disease processes, the rapid accurate detection of biological molecules such as nucleic acids and proteins is becoming more important.

I. Detection of Analytes

The detection of analyte, or “ligand” molecules is an important aspect of current biology, biotechnology, chemistry, and environmental industries. Detection of ligands can be accomplished using many different methods, including the chemical methods of chromatography, mass spectroscopy, nucleic acid hybridization and immunology. Hybridization and immunological methods rely upon the specific binding of ligands to detector, or “receptor” molecules. The basis for specificity of these methods is conferred by a receptor molecule can bind in a specific fashion to the ligand molecule, thereby creating a bound complex. Upon treating the complex under conditions that favor the removal of unbound ligand, the bound ligand can be assayed. The specificity of the binding, the completeness of separating bound and unbound ligands and receptors, and the sensitivity of the detection of the ligand confers the selectivity of the detection system.

For example, in biology and biotechnology industries, analytes such as deoxyribonucleic acid (“DNA”) and messenger ribonucleic acid (“mRNA”) are important indicators of specific genetic, physiological or pathological conditions. DNA can contain important information about the genetic makeup of an organism, and mRNA can be an important indicator of which genes are active in a specific physiological or pathological condition and what proteins may be created as a result of gene activation. Additionally, the direct detection of proteins can be important to the understanding of the physiological or pathological condition of an individual.

DNA is made of a double helix of two strands, each of which is composed of a series or “sequence” of nucleotide bases. The bases found in DNA include adenine, thymine, cytosine and guanine. One strand of the double helix has a sequence of the nucleotides that can be transcribed into mRNA, herein termed a “reading strand,” and the other strand has a sequence of bases, each of which is complementary to the base in the position corresponding in the reading strand. For every adenine in the reading strand, a thymine is present in the other strand. Similarly, for every cytosine in the reading strand, a guanine is present in the other strand. For every guanine and adenine in the reading strand, a cytosine and a thymine, respectively, is found in the other strand. Thus, when the two strands are aligned properly with respect to the other, the complementary bases of each strand can form hydrogen bonds, thereby holding the two strands in a complex, or “hybrid” according to the model of Watson and Crick (“Watson-Crick” hybridization). Thus, the two strands are considered herein to be “complementary” to each other. Ribonucleic acid has a similar structure as DNA, except that thymine is typically replaced by the base uracil. However, uracil is complementary to adenine, and thus, hybridization of RNA can occur with DNA. Because the information content of nucleic acids resides significantly in the sequence of the units that make up the nucleic acid, purely chemical methods that can detect only the presence of nucleotide bases are of limited usefulness. Thus, methods for detecting the presence of specific DNA or RNA relies upon the characterization of the sequence of bases of that nucleic acid.

A. Hybridization Detection of Nucleic Acids

Many different methods are currently in use for the detection of nucleic acids and proteins, but those methods can be time-consuming, expensive, or poorly reproducible. For example, the detection of specific nucleic acid sequences in DNA or RNA molecules can be accomplished using hybridization reactions, wherein an analyte DNA or RNA molecule is permitted to attach to a complementary sequence of DNA. A complementary DNA molecule can be attached to a supporting matrix, and the bound DNA and matrix is herein termed a “substrate.” Exposing an analyte nucleic acid to a complementary substrate DNA can result in the formation of a relatively stable hybrid. Detection of the duplex DNA hybrid is characteristically carried out using methods that can detect labeled DNA analytes. The labeling is typically performed using radioactive, spin resonance, chromogenic or other labels, which are attached to the analyte molecules. Thus, when the labeled analyte attaches to the substrate, unbound analyte can be removed and the bound, or specific, analyte can be detected and quantified.

For example, to detect a mRNA molecule having a specific sequence using current methods, naturally occurring, or “native” mRNA is typically converted to a complementary DNA (“cDNA”) molecule using an enzyme called “reverse transcriptase” under conditions that incorporate a labeled nucleotide into the cDNA. Upon binding of the labeled cDNA to the hybridization substrate, the bound ligand can be detected using a radiometric technique such as scintillation counting, fluorescence or spin resonance, depending on the type of label used.

Currently available methods for the detection of nucleic acids and proteins have undesirable characteristics. The methods are time consuming, require expensive equipment and reagents, require expert manual operations, and the reagents can be environmentally hazardous. Additionally, for assaying mRNA, the methods also can be sensitive to defects in the fidelity of reverse transcription. Unless the cDNA made during reverse transcription is exactly complementary to the mRNA, the analyte will not have the same sequence as the native mRNA, and misleading results can be obtained. The amplification of nucleic acid sequences by the polymerase chain reaction (“PCR”) has been used to increase the numbers of nucleic acid molecules (complementary DNA or “cDNA”) that can be detected. PCR requires DNA polymerase enzymes to amplify the cDNA. Some DNA polymerases can insert incorrect bases into a growing strand of newly synthesized cDNA. In addition, the recognition of ceratin cDNA by DNA polymerase and primers used for PCR can vary depending on the specific sequences of DNA in the sample to be amplified. This variation can result in non-proportional amplification of different cDNA molecules. Subsequent amplification of an strand having an incorrect sequence can result in the presence of several different cDNA sequences in the same sample. Thus, the accuracy and sensitivity of analysis of cDNA using PCR can be compromised.

Additionally, for medical diagnostic or forensic purposes, it can be very important for results of tests to be available rapidly. Commonly used methods for detection of specific nucleic acid sequences can be too slow for therapeutic or forensic uses. Thus, there is a need for rapid, accurate measurement of nucleic acid sequences.

II. Raman Spectroscopy

Raman spectroscopy involves the use of electromagnetic radiation to generate a signal in an analyte molecule. Raman spectroscopic methods have only recently been developed to the point where necessary sensitivity is possible. Raman spectroscopic methods and some ways of increasing the sensitivity of Raman spectroscopy are described herein below.

A. Raman Scattering

According to a theory of Raman scattering, when incident photons having wavelengths in the near infrared, visible or ultraviolet range illuminate a certain molecule, a photon of that incident light can be scattered by the molecule, thereby altering the vibrational state of the molecule to a higher or a lower level. The vibrational state of a molecule is characterized by a certain type of stretching, bending, or flexing of the molecular bonds. The molecule can then spontaneously return to its original vibrational state. When the molecule returns to its original vibrational state, it can emit a characteristic photon having the same wavelength as the incident photon. The photon can be emitted in any direction relative to the molecule. This phenomenon is termed “Raleigh Light Scattering.”

A molecule having an altered vibrational state can return to a vibrational state different from the original state after emission of a photon. If a molecule returns to a state different from the original state, the emitted photon can have a wavelength different from that of the incident light. This type of emission is known as “Raman Scattering” named after C. V. Raman, the discoverer of this effect. If, a molecule returns to a higher vibrational level than the original vibrational state, the energy of the emitted photon will be lower (i.e., have longer wavelength) than the wavelength of the incident photon. This type of Raman scattering is termed “Stokes-shifted Raman scattering.” Conversely, if a molecule is in a higher vibrational state, upon return to the original vibrational state, the emitted photon has a lower energy (i.e., have a shorter wavelength). This type of Raman scattering is termed “anti-Stokes-shifted Raman scattering.” Because many more molecules are in the original state than in an elevated vibrational energy state, typically the Stokes-shifted Raman scattering will predominate over the anti-Stokes-shifted Raman scattering. As a result, the typical shifts of wavelength observed in Raman spectroscopy are to longer wavelengths. Both Stokes and anti-Stokes shifts can be quantitized using a Raman spectrometer.

B. Resonance Raman Scattering

When the wavelength of the incident light is at or near the frequency of maximum absorption for that molecule, absorption of a photon can elevate both the electrical and vibrational states of the molecule. The efficiency of Raman scattering of these wavelengths can be increased by as much as about 108 times the efficiency of wavelengths substantially different from the wavelength of the absorption maximum. Therefore, upon emission of the photon with return to the ground electrical state, the intensity of Raman scattering can be increased by a similar factor.

C. Surface Enhanced Raman Scattering

When Raman active molecules are excited near to certain types of metal surfaces, a significant increase in the intensity of the Raman scattering can be observed. The increased Raman scattering observed at these wavelengths is herein termed “surface enhanced Raman scattering.” The metal surfaces that exhibit the largest increase in Raman intensity comprise minute or nanoscale rough surfaces, typically coated with minute metal particles. For example, nanoscale particles such as metal colloids can increase intensity of Raman scattering to about 10⁶times or greater, than the intensity of Raman scattering in the absence of metal particles. This effect of increased intensity of Raman scattering is termed “surface enhanced Raman scattering.”

The mechanism of surface enhanced Raman scattering is not known with certainty, but one factor can affect the enhancement. Electrons can typically exhibit a vibrational motion, termed herein “plasmon” vibration. Particles having diameters of about 1/10th the wavelength of the incident light can contribute to the effect. Incident photons can induce a field across the particles, and thereby can alter the movement of mobile electrons in the metal. As the incident light cycles through its wavelength, the induced motion of electrons can follow the light cycles, thereby creating an oscillation of the electron within the metal surface having the same frequency as the incident light. The electrons' motion can produce a mobile electrical dipole within the metal particle. When the metal particles have certain configurations, incident light can cause groups of surface electrons to oscillate in a coordinated fashion, thereby causing constructive interference of the electrical field so generated, creating an area herein termed a “resonance domain.” The enhanced electric field due to such resonance domains therefore can increase the intensity of Raman scattering and thereby can increase the intensity of the signal detected by a Raman spectrometer.

The combined effects of surface enhancement and resonance on Raman scattering is termed “surface enhanced resonance Raman scattering.” The combined effect of surface enhanced resonance Raman scattering can increase the intensity of Raman scattering by about 10^{″} or more. It should be noted that the above theories for enhanced Raman scattering may not be the only theories to account for the effect. Other theories may account for the increased intensity of Raman scattering under these conditions.

D. Raman Methods for Detection of Nucleic Acids and Proteins

Several methods have been used for the detection of nucleic acids and proteins. Typically, an analyte molecule can have a reporter group added to it to increase the ability of an analytical method to detect that molecule. Reporter groups can be radioactive, flourescent, spin labeled, and can be incorporated into the analyte during synthesis. For example, reporter groups can be introduced into cDNA made from mRNA by synthesizing the DNA from precursors containing the reporter groups of interest. Additionally, other types of labels, such as rhodamine or ethidium bromide can intercalate between strands of bound nucleic acids in the assay and serve as reporter groups of hybridized nucleic acid oligomers.

In addition to the above methods, several methods have been used to detect nucleic acids using Raman spectroscopy. Vo-Dinh, U.S. Pat. No. 5,814,516; Vo-Dinh, U.S. Pat. No. 5,783,389; Vo-Dinh, U.S. Pat. No. 5,721,102; Vo-Dinh, U.S. Pat. No. 5,306,403. These patents are herein incorporated fully by reference. Recently, Raman spectroscopy has been used to detect proteins. Tarcha et al., U.S. Pat. No. 5,266,498; Tarcha et al., U.S. Pat. No. 5,567,628, both incorporated herein fully by reference, provide an analyte that has been labeled using a Raman active label and an unlabeled analyte in the test mixture. The above-described methods rely upon the introduction of a Raman active label, or “reporter” group, into the analyte molecule. The reporter group is selected to provide a Raman signal that is used to detect and quantify the presence of the analyte.

By requiring reporter groups to be introduced into the analyte, additional steps and time are required. Additionally, the above methods can require extensive washing of the bound and unbound Raman labeled analytes to provide the selectivity and sensitivity of the assay. Moreover, because specific Raman labels must be provided for each type of assay system used, properties of the analytes must be determined in advance of the assay.

### SUMMARY OF THE INVENTION

Thus, one object of this invention is the development of spectroscopic methods that do not rely on labeling of analyte molecules.

Another object of this invention is the development of methods for manufacturing and the manufacture of particle structures for optical detection methods including fluorescence, SERS and SERRS.

These and other objects are met by the design and manufacture of compositions and methods useful for the direct detection of Raman and/or other signals involving electromagnetic radiation. In general, compositions useful for analyte detection of the present invention can use particle structures that are designed to enhance electromagnetic signals, including Raman signals. Particle structures may be fractal, random or ordered.

In certain embodiments of this invention, particle structures can be generated using chemical methods using linkers. Such linked particle structures can be designed and manufactured to have desired properties, including but not limited to selection of wavelengths of incident electromagnetic radiation that permit the generation of enhanced Raman signals to permit sensitive detection of a variety of analytes.

In certain embodiments of this invention, Raman and other electromagnetic signals can be detected for analytes without the need for incorporation of electromagnetically active labels into analyte molecules. Methods of these embodiments as used for Raman spectroscopic methods are herein termed “reverse Raman spectroscopy” or “RRS.” Upon binding of the analyte to the receptor and removal of unbound analyte, the analyte can provide the detectable Raman signal for detection and/or quantification and/or identification. Thus, to detect nucleic acid sequences, oligonucleotide receptor molecules can be made that have sequences complementary to the specific sequences to be identified, but lack a typical component of the analyte molecule. By way of example only, adenine can be replaced by 2,6,-di-aminopurine (“2, 6 AP”) without adversely affecting the binding of thymine residues in the analyte to the receptor molecule. Similarly, 5-methyluridine or 5(1-propynyl)uridine can replace thymine in a complementary nucleic acid sequence without adversely affecting the binding of adenine in an analyte. Moreover, in other embodiments of this invention, deuterium (D₂O) can be used to replace H₂O in certain synthesis of certain receptors. In yet other embodiments of this invention, peptide nucleic acids (herein termed “PNA”) can be used in place of phosphate- and sugar- containing nucleic acids.

In other embodiments of this invention, detection of an analyte-receptor interaction is by observing an alteration in the Raman signal of the analyte receptor complex in the presence of enhancing structures compared to the Raman signals generated by enhancing structures and either the analyte or the receptor.

By using Raman systems as described, the binding of native molecules having the characteristic Raman signal can be detected and thereby can be easily quantified and analyzed. Therefore, these novel methods provide substantial improvements in speed, reliability and accuracy of the detection of biologically active molecules.

In other embodiments of this invention, surfaces are created that promote the surface enhancement effect of SERS. In other embodiments, Raman enhancing surfaces are made that incorporate receptors locally at resonance domains, thereby increasing the sensitivity of Raman spectroscopic detection.

In yet other embodiments of this invention, systems for analysis of biologically significant moieties are provided, wherein a particle structure, receptor and analyte are exposed to incident electromagnetic radiation, and the Raman spectrum of the complexes are used to detect and/or quantify the amounts of analyte present.

In some embodiments, receptors can be attached to or placed near resonance domains, thereby concentrating the productive signal and increasing the sensitivity of detection of analytes.

Certain embodiments provide for the attachment of receptors to resonance domains selectively, thereby decreasing the effects of analyte-receptor complexes at other locations.

In yet further embodiments of this invention, fractal particle structures can be used to enhance a Raman signal generated in the presence of an analyte, thereby providing methods for detection of signals with increased sensitivity.

Although many of the embodiments are illustrated for Raman spectroscopic detection of analytes, principles of this invention can be used for any detection system involving resonance of electromagnetic radiation, including fluorescence methods.

### BRIEF DESCRIPTION OF THE DRAWINGS

The invention will be described with respect to the particular embodiments thereof. Other objects, features, and advantages of the invention will become apparent with reference to the specification and drawings in which:

FIG. 1 is a drawing depicting particle structures of this invention used for spectroscopy.

FIG. 2 depicts particle structures of this invention that has been subjected to photoaggregation.

FIGS. 3a-3c depict a strategy of this invention for chemically linking particles to form particle structures of this invention.

FIGS. 4a-4d depict a strategy of this invention for linking pairs of particle pairs together using linker molecules, and the manufacture of particle structures of this invention.

FIG. 5 depicts another embodiment of this invention in which the linker groups are comprised of aryl di-isonitrile groups.

FIGS. 6a-6e illustrates a photolithographic method for manufacturing particle structures of this invention.

FIGS. 7a-7b depicts particle structures of this invention as in FIG. 2 additionally having receptors. FIG. 7a depicts two particle structures of this invention having oligonucleotide receptor molecules comprising adenine residues (“A”) attached to resonance domains. FIG. 7b depicts two particle structures of this invention having oligonucleotide receptor molecules, similar to that depicted in FIG. 7a, wherein the oligonucleotide receptor molecules comprises 2,6-diaminopurine (“AP”) residues substituted for adenine residues.

FIG. 8 depicts a portion of a particle structure of this invention having AP-substituted oligonucleotide receptors shown binding to thymine residues (“T”) of analyte molecules. The analyte molecules have adenine residues (“A”) that provide a Raman or other electromagnetic signal for detection.

FIG. 8b depicts a matrix of this invention, having defined areas thereon with particle structures within each area.

FIGS. 9a-9b are graphs illustrating the principle of this invention involving the use of an oligonucleotide receptor not having adenine in Raman spectroscopic detection of oligonucleic acids that contain adenine.

FIG. 9c is a graph showing the Raman spectrum of guanine.

FIGS. 10a-10c depict a methods for manufacturing nested particle structures of this invention. FIG. 10a depicts two particles having complementary oligonucleic acid sequences aligned to hold the particles in relationship with each other. FIG. 10b depicts a first-order nested particle structure of this invention. FIG. 10c depicts a second-order nested particle structure of this invention.

FIGS. 11a-11g depict methods for manufacturing biochips of this invention.

FIG. 11a depicts a substrate used for subsequent attachment of particle structures. FIG. 11b depicts a substrate as in FIG. 11a having thiol groups.

FIG. 11c depicts particles of different sizes used to manufacture particle structures of this invention.

FIG. 11d depicts a group of nested particle structures of this invention.

FIG. 11e depicts a group of chemically linked particle structures of this invention.

FIG. 11f depicts a portion of a biochip of this invention having nested particle structures as in FIG. 11d attached to a substrate.

FIG. 11g depicts a portion of a biochip of this invention having chemically linked particle structures as in FIG. 11e attached to a substrate.

FIGS. 12a-12d depict embodiments of this invention having chemically linked particle structures and/or rods.

FIG. 12a depicts two rods useful for enhancement of electromagnetic signals.

FIG. 12b depicts a rod as shown in FIG. 12a having analyte receptors.

FIG. 12c depicts a portion of a biochip of this invention having rods with analyte receptors applied to a substrate.

FIG. 12d depicts a portion of a biochip of this invention having rods with receptors and chemically linked particles structures of this invention applied to a substrate.

FIGS. 13a-13b depict alternative embodiments of this invention. FIG. 13a depicts a top view of a portion of a biochip of this invention having rods/receptors aligned end-to end and within channels inscribed in a substrate, with and without particles. FIG. 13b depicts a cross-sectional view through a portion of a biochip of this invention as described in FIG. 13a.

FIGS. 14a-14d depict the manufacture of a substrate of this invention having a gold surface, silver fractal structures, receptors, and analytes attached thereto.

FIGS. 15a-15d depict Raman spectra of preparations of fractal aggregates of this invention having reduced glutathione and glutathione S-transferase attached thereto.

FIGS. 16a-16e depict Raman spectral enhancement of DTP by fractal structures of this invention.

FIGS. 17a-17c depicts Raman spectra of rhodamine.

### DETAILED DESCRIPTION OF THE INVENTION

Definitions

The following words and terms are used herein.

The term “analyte” as used herein means molecules, particles or other material whose presence and/or amount is to be determined. Examples of analytes include but are not limited to deoxyribonucleic acid (“DNA”), ribonucleic acid (“RNA”), amino acids, proteins, peptides, sugars, lipids, glycoproteins, cells, sub-cellular organelles, aggregations of cells, and other materials of biological interest.

The term “fractal” as used herein means a structure comprised of elements, and having a relationship between the scale of observation and the number of elements, i.e., scale-invariant. By way of illustration only, a continuous line is a 1-dimensional object. A plane is a two-dimensional object and a volume is a three-dimensional object. However, if a line has gaps therein, and is not a continuous line, the dimension is less than one. For example, if ½ of the line is missing, then the fractal dimension is ½. Similarly, if points on a plane are missing, the fractal dimension of the plane is between one and 2. If ½ of the points on the plane are missing, the fractal dimension is 1.5. Moreover, if ½ of the points of a solid are missing, the fractal dimension is 2.5. In scale invariant structures, the structure of objects appears to be similar, regardless of the size of the area observed. Thus, fractal structures are a type of ordered structures, as distinguished from random structures, which are not ordered.

The term “fractal associate” as used herein, means a structure of limited size, comprising at least about 100 individual particles associated together, and which demonstrates scale invariance within an area of observation limited on the lower bound by the size of the individual particles comprising the fractal associate and on the upper bound by the size of the fractal associate.

The term “fractal dimension” as used herein, means the exponent D of the following equation: N∝R^{D}, where R is the area of observation, N is the number of particles, and D is the fractal dimension. Thus in a non-fractal solid, if the radius of observation increases by 2-fold, the number of particles observed within the volume increases by 2³. However, in a corresponding fractal, if the radius of observation increases by 2-fold, the number of particles observed increases by less than 2³.

The term “fractal-derivatized substrate as used herein means a substrate having fractal particle associates, fractal associates or enhancing structures thereon.

The term “fractal particle associates” as used herein means a large number of particles arranged so that the number of particles per unit volume (the dependent variable) or per surface unit changes non-linearly with the scale of observation (the independent variable).

The term “enhancing structure” as used herein means a structure that increases the amplitude of Raman signals generated by an analyte that is near the enhancing structure. The term enhancing structures includes fractal particle associates.

The term “label” as used herein means a moiety having a physicochemical characteristic distinct from that of other moieties that permit determination of the presence and/or amount of an analyte of which the label is a part. Examples of labels include but are not limited to fluorescence, spin-resonance, radioactive moieties. Also known as reporter group.

The term “linker” as used herein means an atom, molecule, moiety or molecular complex having two or more chemical groups capable of binding to a surface and permitting the attachment of particles together to form groups of particles. The simplest linker connects two particles. A branched linker may link together larger numbers of particles.

The term “ordered structures” as used herein means structures that are non-random.

The term “particle structures” as used herein means a group of individual particles that are associated with each other in such a fashion as to permit enhancement of electric fields in response to incident electromagnetic radiation. Examples of particles include metals, metal-coated polymers and fullerenes. Also included in the meaning of the term “particle structures” are films or composites comprising particles on a dielectric surface or imbedded in a dielectric material.

The term “percolation point” as used herein means a point in time on a conductive surface or medium when the surface exhibits an increase in conductance, as measured either via surface or bulk conductance in the medium. One way to measure surface or “sheet” conductance is via electric probes applied to the surface.

The term “Raman array reader” as used herein means a device having a light source and a light detector.

The term “Raman signal” as used herein means a Raman spectrum or portion of Raman spectrum.

The term “Raman spectral feature” as used herein means a value obtained as a result of analysis of a Raman spectrum produced for an analyte under conditions of detection. Raman spectral features include, but are not limited to, Raman band frequency, Raman band intensity, Raman band width, a ratio of band widths, a ratio of band intensities, and/or combinations the above.

The term “Raman spectroscopy” as used herein means a method for determining the relationship between intensity of scattered electromagnetic radiation as a function of the frequency of that electromagnetic radiation.

The term “Raman spectrum” as used herein means the relationship between the intensity of scattered electromagnetic radiation as a function of the frequency of that radiation.

The term “random structures” as used herein means structures that are neither ordered nor fractal. Random structures appear uniform regardless of the point and scale of observation, wherein the scale of observation encompasses at least a few particles.

The term “receptor” as used herein means a moiety that can bind to or can retain an analyte under conditions of detection.

The term “resonance” as used herein means an interaction with either incident, scattered and/or emitted electromagnetic radiation and a surface having electrons that can be excited by the electromagnetic radiation and increase the strength of the electric field of the electromagnetic radiation.

The term “resonance domain” as used herein means an area within or in proximity to a particle structure in which an increase in the electric field of incident electromagnetic radiation occurs.

The term “reporter group” as used herein means label.

The term “reverse Raman spectroscopy” (“RRS”) as used herein means an application of Raman spectroscopy in which an analyte is distinguished by the presence of a Raman spectral feature that is not found in a receptor for that analyte or in the medium in which the analysis is performed.

The term “scaling diameter” as used herein means a relationship between particles in a nested structure, wherein there is a ratio (scaling ratio) of particle diameters that is the same, regardless of the size of the particles.

The term “surface enhanced Raman spectroscopy” (“SERS”) as used herein means an application of Raman spectroscopy in which intensity of Raman scattering is enhanced in the presence of an enhancing surface.

The term “surface enhanced resonance Raman spectroscopy” (“SERRS”) as used herein means an application of Raman spectroscopy in which Raman signals of an analyte are enhanced in the presence of an enhancing surface (see SERS) and when an absorption band of the analyte overlaps with the wavelength of incident electromagnetic radiation.

### EMBODIMENTS OF THE INVENTION

The methods and compositions of this invention represent improvements over the existing methods for spectroscopic methods for detection and quantification of analyte molecules. In particular, the compositions and methods can be desirable for use in conjunction with infrared spectroscopy, fluorescence spectroscopy, surface plasmon resonance, Raman spectroscopy, mass spectroscopy or any other method utilizing excitation of an analyte by electromagnetic radiation.

Certain embodiments of this invention are based upon Surface Enhanced Raman Spectroscopy (“SERS”), Surface Enhanced Resonance Raman Spectroscopy (“SERRS”) and Reverse Raman Spectroscopy (“RRS”). This invention includes methods for manufacturing Raman active structures having specific analyte receptor molecules attached to those structures. The invention also includes methods for detecting analytes using Raman spectroscopy, reverse Raman spectroscopy, compositions useful for reverse Raman spectroscopy, and arrays and test kits embodying Raman spectroscopic methods.

The structures that are desirable for use according to the methods of this invention include structures of small particles in structures, herein termed particle structures, which includes as a subset, fractal associates. Particle structures can be characterized by having physical and chemical structures that enable oscillations of electrons to be in resonance with incident and outgoing electromagnetic radiation.

I. Manufacture of Particle Structures

The Raman active structures desirable for use according to this invention can include any structure in which Raman signals can be amplified. The following discussion regarding metal fractal structures is not intended to be limiting to the scope of the invention, but is for purposes of illustration only.

A. Manufacture of Metal Particles

To make metal particles for nanoscale arrays of receptors according to some embodiments of this invention, we can generally use methods known in the art. Tarcha et al., U.S. Pat. No. 5,567,628, incorporated herein fully by reference. Metal colloids can be composed of noble metals, specifically, elemental gold or silver, copper, platinum, palladium and other metals known to provide surface enhancement. In general, to make a metal colloid, a dilute solution containing the metal salt is chemically reacted with a reducing agent. Reducing agents can include ascorbate, citrate, borohydride, hydrogen gas, and the like. Chemical reduction of the metal salt can produce elemental metal in solution, which combine to form a colloidal solution containing metal particles that are relatively spherical in shape.

### EXAMPLE 1

### Manufacture of Gold Colloid and Fractal Structures

In one embodiment of this invention, a solution of gold nuclei is made by preparing a 0.01% solution of NaAuCl₄ in water under vigorous stirring. One milliliter (“ml”) of a solution of 1% sodium citrate is added. After 1 minute of mixing, 1 ml of a solution containing 0.075% NaBH₄ and 1% sodium citrate is added under vigorous stirring. The reaction is permitted to proceed for 5 minutes to prepare the gold nuclei having an average diameter of about 2 nm). The solution containing the gold nuclei can be refrigerated at 4° C. until needed. This solution can be used as is, or can be used to produce particles of larger size (e.g., up to about 50 nm diameter), by rapidly adding 30 μl of the solution containing gold nuclei and 0.4 ml of a 1% sodium citrate solution to the solution of 1% HAuCl₄.3H₂O diluted in 100 ml H₂O, under vigorous stirring. The mixture is boiled for 15 minutes and is then cooled to room temperature. During cooling, the particles in the solution can form fractal structures. The resulting colloid and/or fractal particle structures can be stored in a dark bottle.

Deposition of enhancing particles on dielectric surfaces including glass can generate films that can enhance electromagnetic signals. Such films can be as thin as about 10 nm. In particular, the distribution of electric field enhancement on the surface of such a film can be uneven. Such enhancing areas are resonance domains. Such areas can be particular useful for positioning receptors for analyte binding and detection. For films or particle structures embedded in dielectric materials, one way to manufacture enhancing structures is to treat the surface until “percolation points” appear. Methods for measuring sheet resistance and bulk resistance are well known in the art.

### EXAMPLE 2

### Manufacture of Metal Particles and Fractal Structures Using Laser Ablation

In addition to liquid phase synthesis described above, laser ablation is used to make metal particles. A piece of metal foil is placed in a chamber containing a low concentration of a noble gas such as helium, neon, argon, xenon, or krypton. Exposure to the foil to laser light or other heat source causes evaporation of the metal atoms, which, in suspension in the chamber, can spontaneously aggregate to form fractal or other particle structures as a result of random diffusion. These methods are well known in the art.

B. Manufacture of Films Containing Particles

To manufacture substrates containing metal colloidal particles of one embodiment of this invention, the colloidal metal particles can be deposited onto quartz slides as described in Examples 1 or 2. Other films can be made that incorporate random structures or non-fractal ordered structures in similar fashions.

### EXAMPLE 3

### Manufacture of Quartz Slides Containing Gold Fractal Structures

Quartz slides (2.5 cm×0.8 cm×0.1 cm) are cleaned in a mixture of HCl:HNO₃ (3:1) for several hours. The slides are then rinsed with deionized H₂O (Millipore Corporation) to a resistance of about 18 MΩ and then with CH₃OH. Slides are then immersed for 18 hours in a solution of aminopropyltrimethoxysilane diluted 1:5 in CH₃OH. The slides are then rinsed extensively with CH₃OH (spectrophotometric grade) and deionized H₂O prior to immersion into colloidal gold solution described above. The slides are then immersed in the gold colloid solution above. During this time, the gold colloid particles can deposit and can become attached to the surface of the quartz slide. After 24 hours, colloid derivatization is complete. Once attached, the binding of colloidal gold nanocomposites to the quartz surfaces is strong and is essentially irreversible. During the procedure, ultraviolet and/or visual light absorbance spectra of such derivatized slides are used to assess the quality and reproducibility of the derivatization procedure. The manufacturing process is monitored using electron microscopy to assess the density of the colloidal coating, the distribution of gold colloid particles on the surface, and the size of the gold colloid particles.

In other embodiments, fractal aggregates can be attached to substrates having a layer of gold metal thereon. The types of substrates are not limited, and can be quartz, conventional glass, plastic or any other substrate upon which a layer of gold metal can adhere. Because gold metal is relatively chemically inert, once prepared, the gold-coated slides can be cleaned using conventional methods prior to attachment of fractal aggregates thereto. Gold or silver fractal aggregates can be prepared using methods described herein or using methods from the prior art. Colloidal of fractal aggregates can then be applied to the surface of the gold-coated substrate and the fractal aggregates tend to adhere to the gold surface, forming a fractal-derivatized or colloidal derivatized substrate.

After attachment of fractal aggregates to the gold surface of the substrate, the fractal-derivatized substrate can be washed to remove unbound colloids. For example, for gold fractal aggregates, it can be desirable to wash the substrate with a solution containing an acid. In certain embodiments, it can be desirable to use nitric acid, and in other embodiments, it can be desirable to use concentrated nitric acid for a period of several hours at a temperature above the freezing point of the acid solution, up to the melting temperature of the fractal aggregates. In other embodiments, other acids can be used, such as HCl, sulfuric acid, acetic acid or other acid. Furthermore the conditions of the washing can be determined by methods known in the art. In certain embodiments, it can be desirable to use a mixture of an acid and an organic solvent, such as acetone to remove materials soluble in such solvents. The types of acids and organic solvents can be selected depending on the types of reagents or contaminants present in the solutions used to prepare the fractal aggregates.

C. Aggregation of Particles to Form Particle Structures

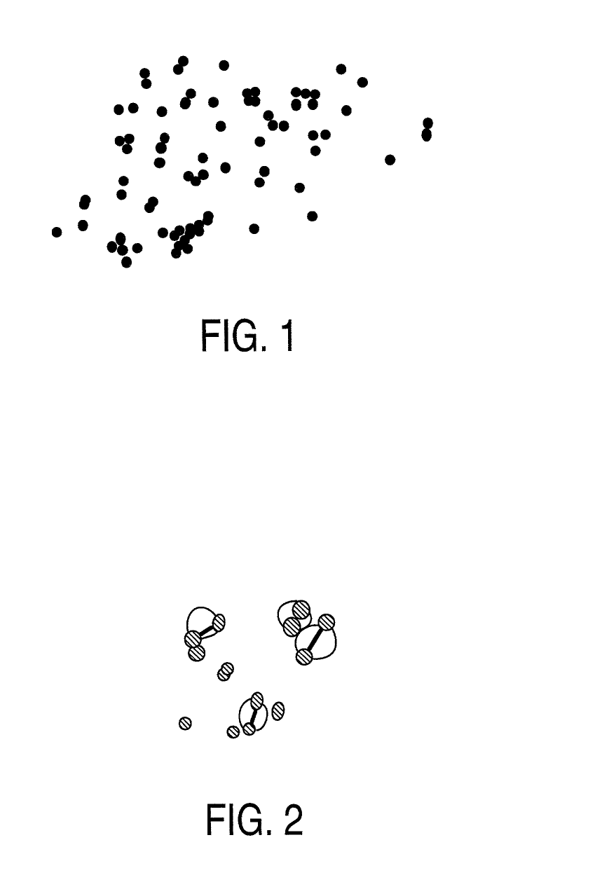

According to other embodiments of this invention, several methods can be used to form particle structures. It is known that metal colloids can be deposited onto surfaces, and when aggregated can form fractal structures having a fractal dimension of about 1.8. Safonov et al., Spectral Dependence of Selective Photomodification in Fractal Aggregates of Colloidal Particles, Physical Review Letters 80(5):1102-1105 (1998) incorporated herein fully by reference. FIG. 1 depicts a particle structure suitable for use with the methods of this invention. The particles are arranged in a scale-invariant fashion, which promotes the formation of resonance domains upon illumination by laser light.

In addition to fractal structures, ordered non-fractal structures and random structures can be generated. These different types of structures can have desirable properties for enhancing signals associated with detection of analytes using electromagnetic radiation.

To make ordered non-fractal structures, one can use, for example, chemical linkers having different lengths sequentially as described in more detail below. In addition, using linkers of the same size, one can generate ordered structures, which can be useful for certain applications.

In certain embodiments of this invention, particles can be attached together to form structures having resonance properties. In general, it can be desirable to have the particles being spheres, ellipsoids, or rods. For ellipsoidal particles, it can be desirable for the particles to have a long axis (x), another axis (y) and a third axis (z). In general, it can be desirable to have x be from about 0.05 to about 1 times the wavelength (λ) of the incident electromagnetic radiation to be used. For rods, it can be desirable for x to be less than about 4λ, alternatively, less than about 3λ, alternatively less than about 2λ, in other embodiments, less than about 1λ, and in yet other embodiments, less than about ½λ. The ends of the rods can be either flat, tapered, oblong, or have other shape that can promote resonance.

For two particle structures, it can be desirable for the particle pair to have an x dimension to be less than about 4λ, alternatively, less than about 3λ, alternatively less than about 2λ, in other embodiments, less than about 1λ, and in yet other embodiments, less than about ½λ.

For two-dimensional structures, pairs of particles, rods, rods plus particles together can be used. The arrangement of these elements can be randomly distributed, or can have a distribution density that is dependent upon the scale of observation in a non-linear fashion.

In other embodiments, rods can be linked together end-to end to form long structures that can provide enhanced resonance properties.

For three-dimensional structures, one can use regular nested particles, or chemical arrays of particles, associated either by chemical linkers in a fractal structure or in ordered, nested arrays.

In yet other embodiments, of third-order structures, a suspension of particles can be desirable. In certain of these embodiments, the suspended particles can have dimensions in the range of about ½λ to about 1 millimeter (mm).

Using the strategies of this invention, a researcher or developer can satisfy many needs, including, but not limited to selecting the absorbance of electromagnetic radiation by particle elements, the nature of the surface selected, the number of resonance domains, the resonance properties, the wavelengths of electromagnetic radiation showing resonance enhancement, the porosity of the particle structures, and the overall structure of the particle structures, including, but not limited to the fractal dimensions of the structure(s).

### 1. Photoaggregation

Photoaggregation can be used to generate particle structures that have properties which can be desirable for use in Raman spectroscopy.

Irradiation of fractal metal nanocomposites by a laser pulse with an energy above a certain threshold leads to selective photomodification, a process that can result in the formation of “dichroic holes” in the absorption spectrum near the laser wavelength (Safonov et al., Physical Review Letters 80(5):1102-1105 (1998), incorporated herein fully by reference). Selective photomodification of the geometrical structure can be observed for both silver and gold colloids, polymers doped with metal aggregates, and films produced by laser evaporation of metal targets.

One theory for the formation of selective photomodification is that the localization of optical excitations in fractal structures are prevalent in random nanocomposites. According to this theory, the localization of selective photomodification in fractals can arise because of the scale-invariant distribution of highly polarizable particles (monomers). As a result, small groups of particles having different local configurations can interact with the incident light independently of one another, and can resonate at different frequencies, generating different domains, called herein “optical modes.” According to the same theory, optical modes formed by the interactions between monomers in fractal are localized in domains that can be smaller than the optical wavelength of the incident light and smaller than the size of the clusters of particles in the colloid. The frequencies of the optical modes can span a spectral range broader than the absorption bandwidth of the monomers associated with plasmon resonance at the surface. However, other theories may account for the effects of photomodification of fractal structures, and this invention is not limited to any particular theory for operability.

Photomodification of silver fractal aggregates can occur within domains as small as about 24×24×48 nm³ (Safonov et al., Physical Review Letters 80(5):1102-1105 (1998), incorporated herein fully by reference). The energy absorbed by the fractal medium can be localized in a progressively smaller number of monomers as the laser wavelength is increased. As the energy absorbed into the resonant domains increases, the temperature at those locations can increase. At a power of 11 mJ/cm², light having a wavelength of 550 nm can produce a temperature of about 600 K (Safonov et al., Physical Review Letters 80(5:1102-1105 (1998), incorporated herein fully by reference). At this temperature, which is about one-half the melting temperature of silver, sintering of the colloids can occur (Safonov et al., Id.) incorporated herein fully by reference), thereby forming stable fractal nanocomposites.

As used in this invention, photoaaggregation can be accomplished by exposing a metal colloid on a surface to pulses of incident light having a wavelengths in the range of about 400 nm to about 2000 nm. In alternative embodiments, the wavelength can be in the range of about 450 nm to about 1079 nm. The intensity of the incident light can be in the range of about 5 mJ/cm² to about 20 mJ/cm². In an alternative embodiment, the incident light can have a wavelength of 1079 nm at an intensity of 11 mJ/cm².

Fractal aggregates that are especially useful for the present invention can be made from metal particles having dimensions in the range of about 10 nm to about 100 nm in diameter, and in alternative embodiments, about 50 nm in diameter. A typical fractal structure of this invention is composed of up to about 1000 particles, and an area of the aggregate typically used for large-scale arrays can have a size of about 100 μm×100 μm.

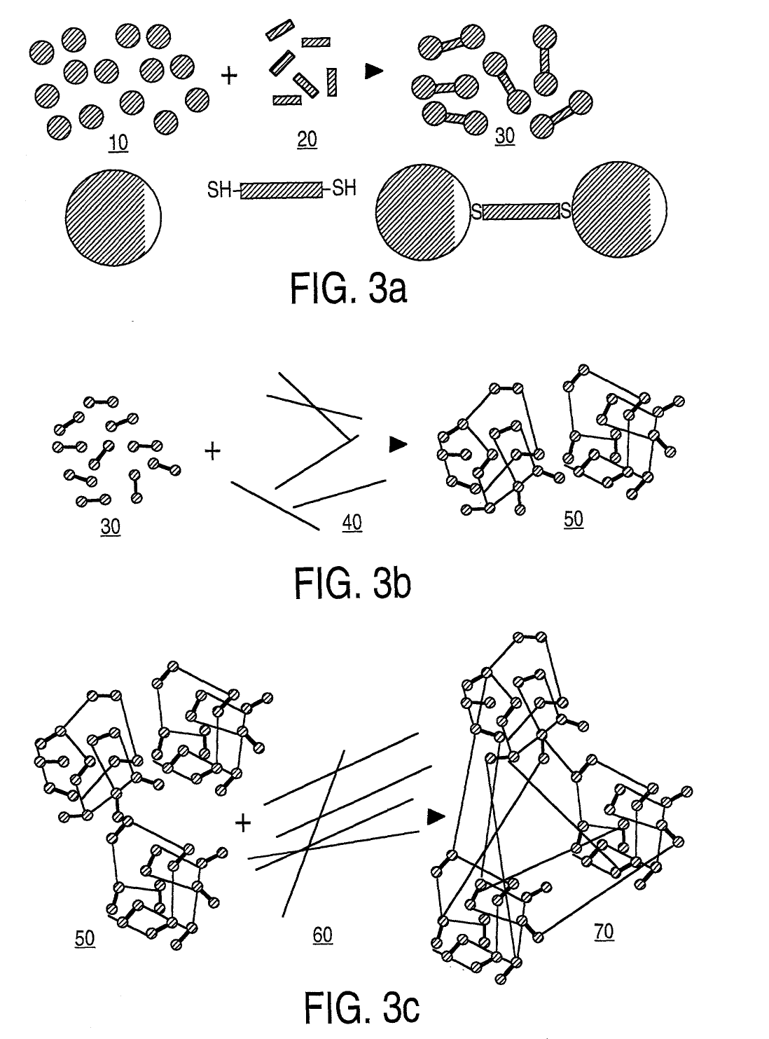

FIG. 2 depicts a particle structure that have been photoaggregated and that are suitable for use with the methods of this invention. Local areas of fusion of the metal particles can be observed (circles).

### 2. Chemically Directed Synthesis of Particle Structures

In certain embodiments of this invention, particle structures can be made using chemical methods. First, metal particles can be either made according to methods described above, or alternatively can be purchased from commercial suppliers (NanoGram Inc., Fremont, Calif.). Second, the particles can be joined together to form first-order structures, for example, pairs of particles. Then, the first-order structures can be joined together to form second-order structures, for example, pairs of particle pairs. Finally, third-order fractal structures can be made by joining second-order structures together.

In alternative embodiments of this invention, the formation of a fractal array of metal particles can be carried out using chemical methods. Once metal colloid particles have been manufactured, each particle can be attached to a linker molecule via a thiol or other type of suitable chemical bond. The linker molecules then can be attached to one another to link adjacent colloid particles together. The distance between the particles is a function of the total lengths of the linker molecules. It can be desired to select a stoichiometric ratio of particles to linker molecules. If too few linker molecules are used, then the array of particles will be too loose or may not form at all. Conversely, if the ratio of linker molecules to particles is too high, the array may become too tight, and may even tend to form crystalline structures, which are not random, and therefore will not tend to promote surface enhanced Raman scattering.

In general, it can be desirable to perform the linking procedure sequentially, wherein the first step comprises adding linker molecules to individual particles under conditions that do not permit cross-linking of particles together. By way of example only, such a linker can comprise an oligonucleotide having a reactive group at one end only. During this first step, the reactive end of the oligonucleotide can bind with a metal particle, thereby forming a first particle-linker species, and having a free end of the linker. The ratio of linker molecules to particles can be selected, depending on the number of linker molecules are to be attached to the particle. A second linker can be attached to another group of particles in a different reaction chamber, thereby resulting in a second linker-particle species, again with the linker having a free end.

After those reactions have progressed, the different linker-particle species can be mixed together and the linkers can attach together to form “particle pairs” joined by the linker molecules.

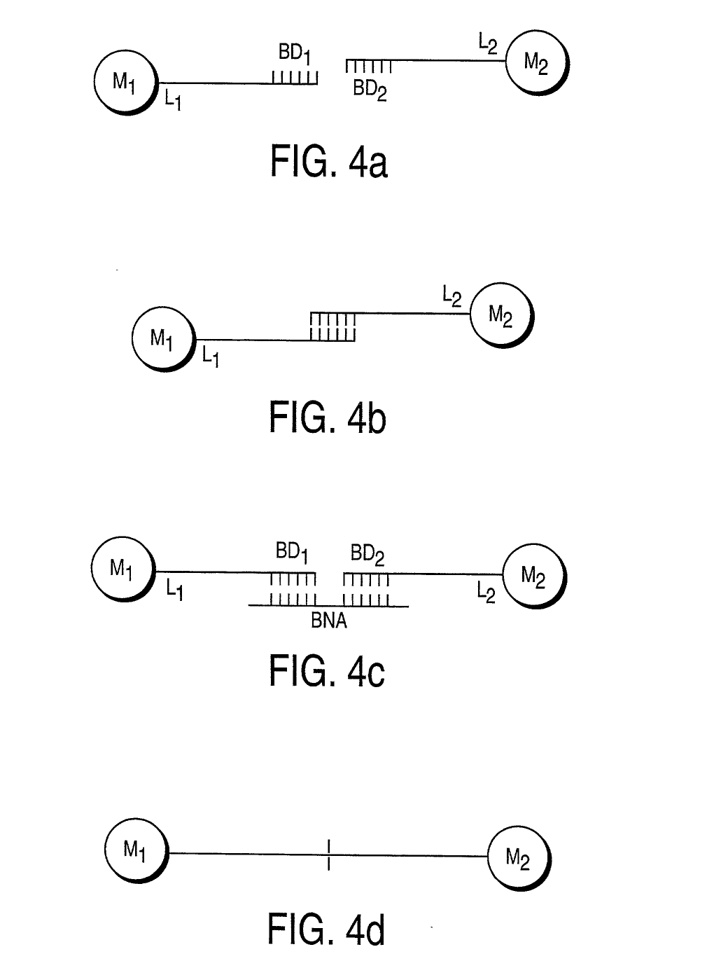

By way of example, FIG. 3a to 3c illustrates methods for manufacturing fractal structures of this invention. In FIG. 3a, metal particles 10 are formed using methods previously described. Short linkers 20 have chemically active ends that are capable of binding to metal particles 10. For example, linker 20 has sulfhydryl (“SH”) groups at each end of the linker 20. When combined, metal particles 10 bind with the SH ends of linkers 20 to form particle pairs 30.

FIG. 3b illustrates the steps that can be used to form clusters of particle pairs. Particle pairs 30 are reacted with medium-length linkers 40 to form clusters 50.

FIG. 3c illustrates the steps that can be used to form nanoscale fractal structures of this invention. Clusters 50 are reacted with long linkers 60 to form nanoscale fractal structure 70.

In other embodiments, nucleic acids can be used as linkers, based upon the ability of DNA to form hybrids with nucleic acids comprising complementary sequences. DNA ligases or other mechanisms can be used to join the linkers together to form a complete linker between metal particles.

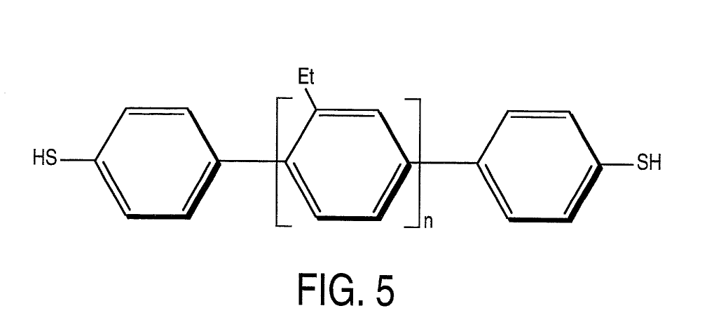

FIG. 4 depicts, in general, the linkage of metal particles to form particle pairs using linkers having binding domains. FIG. 4a depicts two metal particles (M), each having a linker molecule (L1 or L2) having a desired length, comprising inter-linker binding domains (BD1 and BD2). The inter-linker binding domains are unbound. FIG. 4b depicts the particles shown in FIG. 4a after binding of the inter-linker binding domains to form a particle pair. In embodiments in which the linkers are nucleic acids, the binding domains can have complementary sequences, such that the nucleotide residues can form stable hybrid complexes with each other, thereby linking the metal particles together as a pair. In certain embodiments, the sequence of BD1 can be poly[adenine] for example, A₁₀. The sequence of BD2 can be poly[thymidine], for example, T₁₀. Thus, A₁₀ can hybridize to T₁₀, thereby forming a stable hybrid. In other embodiments, the lengths of the binding domains can be any convenient length that permits the formation of a stable hybrid.

In other embodiments, illustrated in FIG. 4c, BD1 and BD2 can be selected to be complementary to a third nucleic acid, herein termed a “bridge nucleic acid” or (“BNA”), comprising two sequences, one complementary to BD1 and an other complementary to BD2. When the BNA is placed in contact with BD1, the portion of BNA complementary to BD1 can form a stable hybrid of the first metal particle M1 with LI and BNA attached thereto. However, the portion of the BNA that is complementary to BD2 of L2 is free to hybridize to BD2. Upon exposure of the M1-L1-BNA complex to the M2-L2, the BD2 can bind to that portion of the BNA complementary to BD2, forming a stable particle pair.

FIG. 4d depicts an alternative particle pair in which the inter-linking molecules are attached by way of their ends. This can be accomplished, for example, by treating the particle pair shown in FIG. 4c with a DNA ligase to form a covalent bond between L1 and L2, and then by digesting away the bridge nucleic acid.

After the pairs of particles are formed, additional linkers can be attached to the particle pairs, and the process can be repeated to form “pairs of particle pairs.”Subsequently, the process can be repeated until 3 or more orders of particle structures are formed. Under these conditions, one can manufacture structures having any desired porosity. In general, the size of the nanoscale structures should have average dimensions in the range of about 20 nm to about 500 nm. In alternative embodiments, the dimensions can be in the range of about 50 nm to about 300 nm, and in other embodiments, in the range of about 100 to about 200 nm, and in yet other embodiments, about 150 nm.

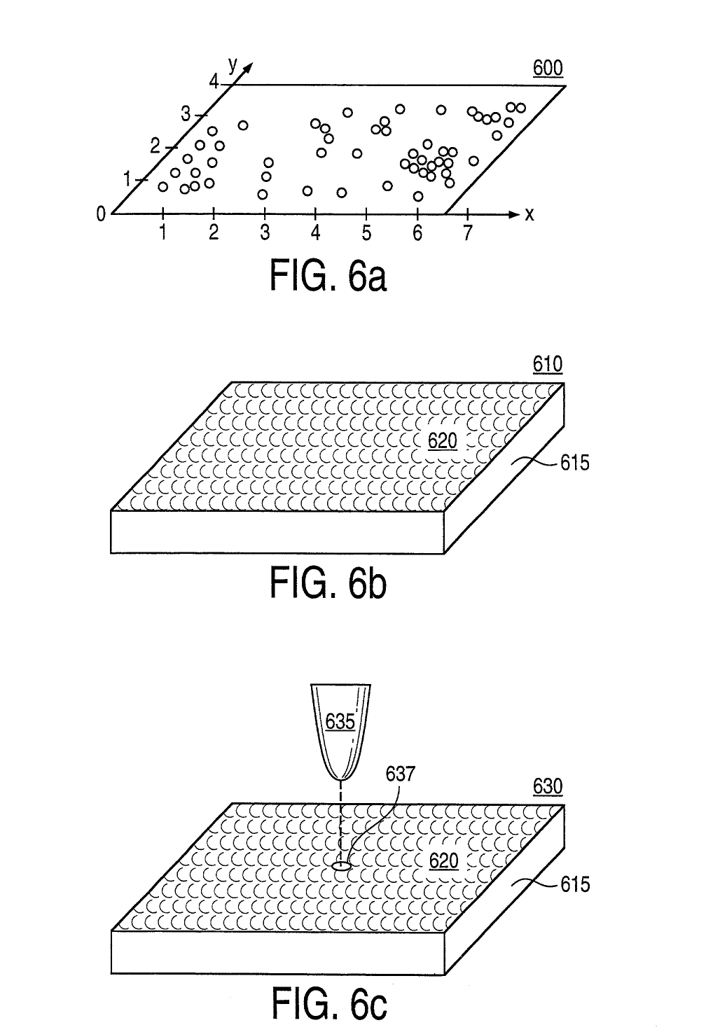

In other embodiments of this invention, the linking can be carried out using an aryl di-thiol or di-isonitrile molecules. FIG. 5 depicts the structure of a class of linkers having thiol (SH) groups at each end. Alternatively one can use any active moiety that can be used to attach the linker to the metal particle. It can be desirable to use the above types of aryl linkers with nucleic acid or other types of linker molecules. The linker can have a central area having ethylbenzene moieties, where n is a number between 1 and about 10,000.

In general, the ratio of length for each subsequent pairs of linkers can be in the range of about 2 to about 20. Alternatively, the ratios of lengths of subsequent pairs of linkers can be in the range of about 3 to about 10, and in other embodiments, about 5. In certain other embodiments, the ratio of linker lengths in successive orders can be nonconstant, thus resulting in the manufacture of an ordered, non-fractal structure.

For example, for a three-order manufacturing process, it can be desirable for the ration of L1:L2:L3 to be in the range of about 1:2:4. Alternatively, the ratio can be about 1:5:25, and in yet other embodiments, the ratio can be about 1:20:400. In other embodiments, the ratio between L1 and L2 and from L2 to L3 need not be the same. Thus, in certain embodiments the ration of L1:L2 :L3 can be 1:3:20, or alternatively, 1:20:40.

### 3. Manufacture of Suspensions of Fractal Particle Associates

In certain other embodiments of this invention, suspensions of fractal particle associates (fractal associates) can be used, for example, to provide a structure in solution that can bind or retain analytes for detection using methods of this invention. The size of fractal particle associates can be in the range of from hundreds of nanometers to mm dimensions. The fractal associates can comprise a number of particles arranged by means of chemical linkers. The number of particles per fractal associate can be as few as about 100 particles, or alternatively, thousands can be used to form a fractal associate. By increasing the number of particles in a fractal associate, the increase in the void size increases by a greater proportion.

### 4. Nested Fractal Structures

In another series of embodiments of this invention, nested fractal structures are provided. Nested fractal structure, for example, comprises a core of a large particle, surrounded by a “halo” of smaller particles, and each of the smaller particles is surrounded by a “halo” of even smaller particles. (See Example 6). Nested fractal structures can be especially useful for generation of essentially uniform fractal surfaces for enhanced analyte detection. It can be desirable to include large excesses of smaller particles compared to larger particles for each successive step. For example, it can be desirable to have excess of smaller particles in the range of about 10 to about 1000 times the number of larger particles. Alternatively, it can be desirable to have an excess of smaller particles of between 10 and 100 times the number of larger particles, and in other embodiments, it can be desirable to have smaller particles in excess of about 10 times the number of larger particles.

### 5. Lithographic Manufacture of Particle Structures

In other embodiments of this invention, particle structures can be manufactured using lithographic methods known in the semiconductor manufacturing arts. To manufacture particle structures, an image of the particle structure to be made can be made and stored in a computer memory. Each point defining the particle structure can be represented by a single location within the memory. The memory device can then direct the projection of a beam of electromagnetic radiation, electrons, or other particles locally onto a suitable surface. The beam can create site on the surface for the subsequent formation of a metal particle at desired locations.

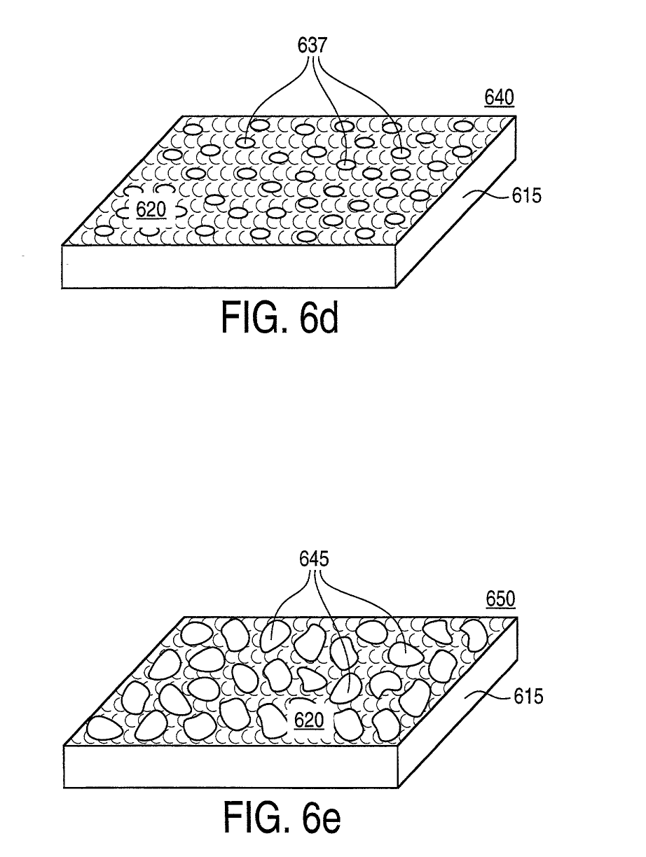

By way of example, such a method is disclosed in Xioa et al, Hunting for the Active Sties of Surface-Enhanced Raman Scattering: A New Strategy Based on Single Silver Particles, J. Physical Chemistry B 101:632-638 (1997), incorporated herein fully by reference. FIG. 6 depicts several steps in the lithographic manufacture of a particle structure of this invention. FIG. 6a shows an image 600 of a desired distribution of nanoparticles. The image is stored in a computer memory, in which each particle is represented by a pair of reference coordinates, one x and one y for each point. FIG. 6b depicts a substrate for nanoparticle structure 610 comprising a gold substrate 615 having a film of hexadecanethiol 620 on which the nanoparticle structure is to be manufactured. FIG. 6c illustrates the placement of the tip 635 of a scanning tunneling microscope (STM) over the gold substrate 615 at a point stored in the computer memory. Electrons emitted from the tip 635 of the STM can interact with the hexadecanethiol film 620 to cause a patch 637 to form, and subsequent etching with cyanide (FIG. 6d) can expose a series of patches 637 in the surface of the underlying gold substrate 615. Thus, the pattern of particle positions stored in the computer's memory can be physically reproduced on the surface of the substrate. Subsequently, silver or other metal can be electrochemically deposited only at those locations 645 where the hexadecanethiol film 620 has been removed, thus forming the nanoparticle structure 650 (FIG. 6e).

Alternatively, traditional semiconductor masks can be used to direct the location of nanoparticle structures on substrates. Regardless of the method used, the result obtained will provide for resonance properties of the structures.

II. Manufacture of Receptor-Derivatized Particle Structures

Once the particle structures of metal particles have been manufactured, receptors can then be attached, thereby forming receptor-derivatized structures that are useful for spectroscopic detection and quantification of analytes.

A. Selection of Receptor

The receptor chosen to be attached to particle structures of this invention will depend on binding properties of the desired analyte. For example, to detect and quantify nucleic acid sequences, it can be desirable to use oligonucleotide receptors. Oligonucleotide receptors can hybridize to analyte nucleotide sequences, thereby producing a bound ligand. Alternatively, if desired, one can use an antibody directed against a nucleotide sequence to bind the nucleic acid. In other embodiments, DNA binding proteins can be used. For example, to detect certain promoter regions of genes, specific promoter-binding proteins can be used as receptors. Moreover, or peptide nucleic acids can be used to bind native nucleic acids.

Similarly, to detect protein analytes, antibodies and other, specific protein binding molecules can be used. Once the type of analyte is chosen, the specific receptor molecule and the conditions for its attachment to the fractal array can be determined. Additionally, antibodies directed against low molecular weight analytes can be attached to a substrate.

By way of example, the nucleic acid receptors can advantageously used in a large scale matrix array to measure a large number of analyte sequences simultaneously.

### EXAMPLE 4

### Synthesis of Receptors of Nucleic Acid Oligomers

Thiol-derivatized DNA oligomers are synthesized by standard phosphoramidite chemistry according to the methods of Caruthers Gene Synthesis Machines: DNA Chemistry and Its Uses, Science 230:281-285 (1995), incorporated herein fully by reference. Such oligomers are obtained from Dr. Keith McKenney of The Institute for Genomic Research (TIGR), Rockville, Md., and are prepared according to the methods of Peterlinz et al. Observation of Hybridization and Dehybridization of Thiol-Tethered DNA Using Two-Color Surface Plasmon Resonance Spectroscopy, Journal American Chemical Society 119:3401-3402 (1997), incorporated herein fully by reference.

The DNA oligomers are selected to be in the range of about 10-50 bases in length, although much longer sequences can also be used. In other embodiments, the DNA oligomers are in the range of about 15-30 bases in length, and in alternative embodiments, the DNA oligomers are about 25 bases in length. If the oligomer is too long, the analyte molecule can be too far from the metal surface, and the surface enhancement of Raman resonance can be undesirably low. If the oligomer is too short, the specificity of hybridization can be too low. Therefore, the length of the oligomer is selected to optimize the sequence specificity and resonance enhancement of the analyte. In situations in which sequence specificity is less important than resonance enhancement, shorter oligomers can be desirable. Conversely, in situations in which a high degree of sequence specificity is desired, longer oligomers can be desirably used.

Two sets of complementary nucleotide oligomers are synthesized, one set being manufactured using moieties that lack a Raman active component. In certain embodiments, the DNA oligomer is synthesized using 2,6 di-aminopurine instead of adenine.

In other embodiments of this invention, peptide nucleic acid (“PNA”) receptors are used. Peptide nucleic acids have an affinity to RNA and DNA comparable to that of DNA, (Griffin (1998); Kyger et al (1998); Igloi (1998); Ratilainen et al. (1998), each reference herein incorporated fully by reference), and thus, can form hybridization pairs with mRNA. The difference between the chemical structures of PNA and DNA can result in a pronounced difference in their Raman spectra. In particular, the bands corresponding to nucleic acid phosphodiester backbone bonds, absent in the PNA attached to a substrate, appear when the PNA is bound to a DNA or mRNA ligand upon hybridization (Guan (1996)). PNA fragments can be obtained from Atom Sciences (Oak Ridge, Tenn.).

B. Attachment of Receptors to Metal Colloid

In general, oligomers can be attached to metal surfaces via an alkanethiol covalently linked at the 5′ position of single-stranded DNA oligomers according to the methods of Heme, Characterization of DNA Probes Immobilized on Gold Surfaces, Journal American Chemical Society 119:8916-8920 (1997), incorporated herein fully by reference. The attachment can be irreversible, thereby permitting hybridization and dehybridization on the surface (Peterlinz et al., Observation of Hybridization and Dehybridization of Thiol-Derivatized DNA Using Two Color Surface Plasmon Resonance Spectroscopy. Journal American Chemical Society 119:3401-3402 (1997), incorporated herein fully by reference). However, any method can be used that results in the attachment of receptor molecules to metal surfaces and can permit the receptor to maintain the physical characteristics necessary for specific binding to ligands.

### EXAMPLE 5

### Linking of DNA to Colloidal Gold

The colloidal gold-coated quartz slides of Example 3 can then used as a matrix or substrate for the binding of DNA used for hybridization detection of analyte nucleic acids.

The gold colloid derivatized slides are placed in 1.0 M KH₂PO₄ buffer solution, pH 3.8, containing 1.0 μM thiol-derivatized DNA for a specific amount of time to achieve thiol-tethering of DNA. The surface is then passivated by exposing the DNA tethered slides to 1.0 mM mercaptohexanol (HS(CH₂)₆OH) for 1 hour. This treatment eliminates nonspecific binding of polynucleotides. Thorough rinsing with deionized water is required before analysis of hybridization.

C. Attachment of Receptors to Resonance Domains

In other embodiments of this invention, receptors can be localized to resonance domains within particle structures. Upon illumination of the particle structures, resonant domains can be heated, and that heating can cause partial melting of the metal particles. Typically, the dimensions of resonance domains are smaller than the wavelength of the incident light. The size of the resonance domains generated at certain wavelengths of incident light can be on the order of 1/25 of the wavelength of the light used in their generation. However, as the wavelength of light becomes longer, the size of the resonance domains can become smaller. Resonant domains are areas that can exhibit intense resonance, and can produce greater amplification of Raman signals than that possible in unaggregated metal or metal colloid substrates. Thus, resonance domains that are especially useful for this invention can be made using incident light, which can result in resonance domains comprising between about 4 to about 10 monomer particles.

In certain embodiments of this invention, the property of particle structures to become locally heated can be used advantageously to localize receptor molecules to those locations. To manufacture a particle structures having localization of resonance domain-specific receptors, a surface containing particle structures is prepared as above. A solution containing receptor molecules is then placed on the surface and in contact with the particle structures. Pulses of laser light are used to illuminate the surface, and at those locations where resonance domains are created, the local temperature of the reaction mixture can reach the threshold for the formation of intermolecular bonds between the particle structures and the receptor, thus attaching the receptor to the particle structures. In general, any thermosensitive chemistry for linking the receptors to the substrate can be used.

Generally, the power required to initiate receptor molecule derivatization is less than that needed for photoaggregation. It can be desirable to provide temperatures at the resonance domains in the range of about 0° C. to about 500° C., alternatively in a range of about 20° C. to about 300° C., in other embodiments, in the range of about 50° C. to about 180° C. In yet other embodiments, the temperature can be in the range of about 70° C. to about 100° C.

The temperature needed will vary with the threshold temperature required to initiate the linkage of the receptor to the metal surface. In certain embodiments, it is desirable that the temperature locally at the resonance domains remain below the temperature at which bond breakage and reversal of the bond between the receptor and the metal surface occurs.

In other embodiments of this invention, photosensitive reagents can be used to link the receptor to the particle structures at specific locations. A number of such reagents can be obtained from Pierce Products Inc., Rockford, Ill. By the use of different photochemical linking agents, one can link different types of receptors to the same substrate. For example, one can attach DNA and proteins to the same substrate.

It can be desirable to limit the attachment of receptor molecules to specific sites on a substrate. This can be accomplished by using wavelengths of light that are relatively short, for example, less than about 1000 nm, in other embodiments, below about 600 nm, in yet other embodiments, below about 400 nm. Also, laser light can be desirable in situations in which the site of attachment is to be localized to areas of high electric field. In this case, it can be desirable to use double- or triple-photon processes, in which multiple photons having long wavelengths can reach the photoreactive moiety on the receptor and particle structure to provide sufficient energy to cause a linking reaction to occur. This can occur even if the energy of a single photon is insufficient to initiate the photochemical reaction.

Once manufactured, receptor molecules localized to the resonance domains of the fractal arrays can remain at those locations during subsequent exposures to incident light.

In other embodiments of this invention, attachment of receptors at resonance domains can be performed using a scanning atomic force microscope (see Hansen et al. “A Technique for Positioning Nanoparticles Using an Atomic Force Microscope”, Nanotechnolozy 9:337-342 (1998), incorporated herein fully by reference). having a capillary tip and optical feedback. In these embodiments, the capillary contains derivatized receptors which can be deposited onto a surface. In the process of deposition, the surface can be illuminated by incident electromagnetic radiation produced by a laser. At resonance domains, the resonance increases the intensity of the emitted radiation and thereby provides a signal to the optical feedback device to initiate deposition of receptors at those locations, depending upon the intensity of electromagnetic radiation emitted from the surface in response to external illumination provided by the laser.

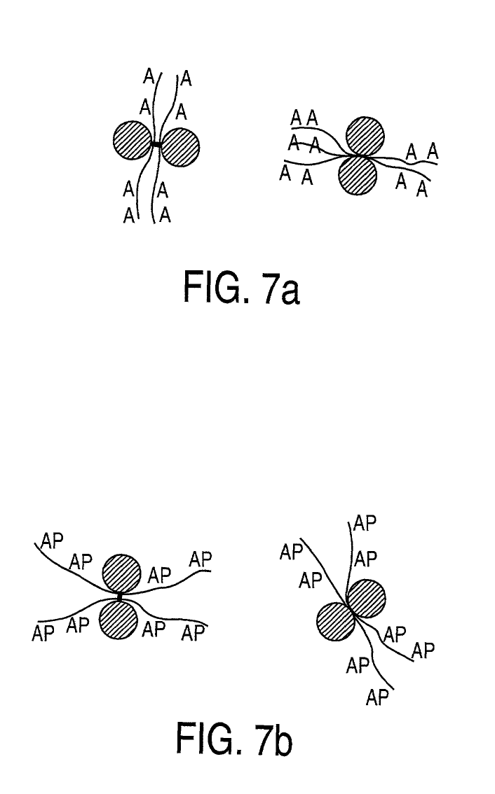

FIGS. 7a and 7b depict embodiments of this invention in which receptor molecules are attached to resonance domains of particle structures. FIG. 7a depicts an area of a particle structures in which the receptor molecules are native, adenine (“A”)-containing oligonucleotides. FIG. 7b depicts a particle structures similar to that shown in FIG. 7a but having the adenine moieties replaced by 2, 6-diaminopurine (“AP”).

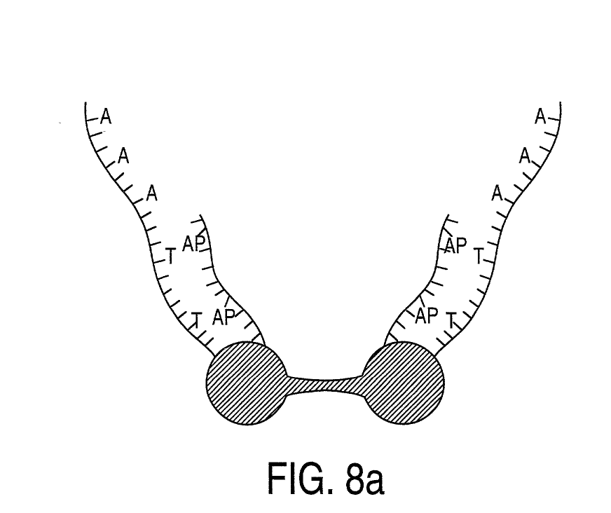

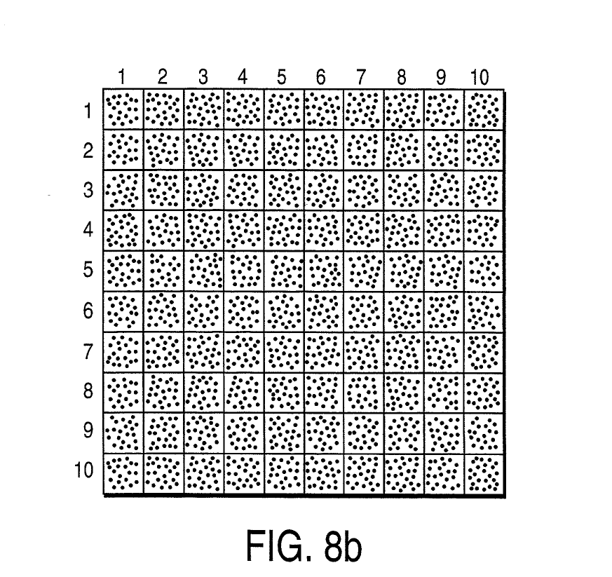

FIG. 8a depicts the binding of native, complementary oligonucleotide analytes to a particle structures containing receptors as shown in FIG. 7b, having adenine replaced by 2, 6-diaminopurine (AP). Analyte molecules containing adenine (A) are depicted as hybridizing to the oligonucleotide receptor such that the adenine residues bind to the 2, 6-diaminopurine residues of the receptor molecule.

III. Design and Manufacture of Matrix Arrays

The processes described above for the derivatization of metal colloid aggregates can be extended to the manufacture of matrix arrays having a large number of different receptors. In such an array, there can be numerous individual defined areas, or “cells” that have a particular type of receptor bound to the metal colloid aggregate. The size of each cell can be on the order of about 100 μm×100 μm. Within each of these cells, a single type of receptor- fractal aggregate can be manufactured. Thus, in a matrix array of 10 cm×10 cm, there can be up to about 10⁶ different cells, each of which can have a different fractal aggregate receptor type.

FIG. 8b depicts an array comprising numerous cells or defined areas, each of which has particle structures containing a plurality of receptors bound to each defined area, and being specific for a desired analyte. The large-scale array shown is a 10×10 matrix, with individual cells positionally located within the large-scale array. Other array configurations can be desirable, and includes arrays having identifier moieties different from the receptor molecules. Identifier moieties can be used to define the position and/or the type of receptor molecule characteristic of the particular defined areas. Such identifier moieties can include nucleic acids of defined sequence, or can include identifiers produced by combinatorial chemical methods known in the art. Moreover, defined areas can be identified using colored markers.

By way of example only, a large-scale array containing fractal aggregates can be exposed to a first receptor type and a beam of highly focused incident light can selectively illuminate one or a few specific cells, thereby linking the first receptor to the substrate in only those cells in which fractal aggregates with the first receptor type is desired. Beams of highly focused laser light having the necessary dimensions can be routinely produced using of photolithography methods used in semiconductor manufacture. Subsequently, the substrate can be washed to remove unbound first receptor type, and a second receptor type can be applied to the substrate. Laser light can illuminate different cells to link the second receptor type to fractal aggregates to form fractal aggregates with the second receptor type. The process of sequential application of any desired number of different receptor types to different cells in the matrix array can be carried out using the same chemistry of linkage if desired, or different types of chemical linkage can be used. The methods above can be fully automated, so that the reproducibility of manufacture of fractal aggregates can be quite high.

A result of this process is that a matrix array containing a large number of positionally identifiable cells can be manufactured. Such arrays can be used to detect and determine sequences of DNA or mRNA, using strategies as described in, for example, U.S. Pat. No. 5,925,525, incorporated herein fully by reference.

IV. Detection of Analytes

Detection of analytes according to methods of this invention includes the use of a Raman reader and a matrix array. Detection can be performed using a pre-manufactured substrate having particle structures atop the substrate. The substrate can have cells or defined areas thereon, having a single type of receptor. When a sample containing an analyte is applied to such a matrix, analytes can bind to or be retained by receptors having sufficient affinity. The matrix can then be washed to remove unbound analytes, leaving only those analytes that have a sufficient affinity for the receptors to which they are bound. The matrix array can then be subjected to analysis using a reader or be performed using a light source focused upon the array, one cell at a time. Light is projected at the cell, and reflected, scattered, or re-emitted light can be collected and transmitted to the light detector. Collected light can be analyzed for Raman spectral features, and such features can be compared with Raman features derived from known moieties. Such known spectra can be imported from external databases, which can include information on biological significance of specific analytes. Analysis of information can be performed using a computer, which can be associated with a memory device for storing a program to carry out spectral analyses. Also, an output device, such as a screen display or a printer can provide information to the user. Such comparison can be the basis for determining the amount of analyte in the cell on the matrix array. Additionally, changes in the analyte due to the conditions of measurement can be determined, and any artifacts, such as non-specific binding so introduced can be discovered.

In other embodiments, detection can be performed under conditions in which resonance of electron transition in analyte molecules does not occur. According to one theory, this situation can be created when the frequency of incident light does not overlap the absorbance band of the analyte. In these situations, it can be desirable to add a suspension of particles atop the substrate and receptor analyte complexes. Enhancement of Raman signals can be sufficient to provide a highly sensitive detection.

In certain other embodiments, a combination of resonance conditions and enhancement provided by particle structures can be desirable to provide high sensitivity.

In yet other embodiments, a Raman array reader can be used to detect and quantify the amount of analyte bound to a cell of a matrix array. A Raman reader can be sued for parallel, rapid and sensitive detection of analytes by acquiring Raman spectral features of each cell of an array and comparing the spectral features with known spectral features. Thus, the existence, identity and amount of an analyte can be determined.

In some embodiments, it can be desirable to use light sources that provide different wavelengths of light simultaneously. These sources can be less expensive and if the wavelengths are sufficiently different from each other, the interference with acquiring unique Raman spectra can be minimized.

Detection of analytes according to some embodiments of this invention is advantageously carried out using native analytes. To carry out such a detection, it can be desirable to use receptor molecules that are lacking a structural feature of the analyte that is responsible for a Raman signal. Such a strategy is termed herein, “Reverse Raman Spectroscopy,” or “RRS.” In general, nucleic acids can be detected advantageously using RRS. Several examples of this strategy follow herein below

A. Modified Nucleic Acid Receptors

Detection of nucleic acid analytes using RRS typically involve the use of receptor molecules that are lacking native nucleotide bases. The nucleotide bases cytosine, uracil, thymine, guanine and adenine each exhibit Raman bands at wavenumbers in the range of about 610 cm⁻¹ to about 800 cm⁻¹. Several nucleotide analogs that have no Raman bands in this range can be suitable for use with the methods and compositions of this invention.

### 1. Substitution of Adenine

The nucleotide adenine is composed of a purine ring structure that has a characteristic Raman scattering band at 733 cm⁻¹ (Kurokawa et al. Surface-Enhanced Raman Spectroscopic Detection of CO₂, SO₃, and Nucleic Acid Bases Using Polyvinyl Alcohol Film Doped with AgFine Particles. Analytic Biochemistry 209:247-250 (1993), incorporated herein fully by reference. If a receptor molecule incorporates adenine as a base to pair with thymine residues according to Watson-Crick base pairing, there will be a large Raman band observed at 733 cm⁻¹ even though no analyte is adsorbed to the receptor, thus, making the resolution of analyte nucleic acids containing adenine difficult. Because most nucleic acids of interest contain adenine, the presence of native adenine in receptors poses a problem.

To overcome this problem, an adenine analog can be incorporated into the receptor nucleic acid sequence in the place of adenine. Any adenine analog that (1) lacks a characteristic Raman band, and (2) can bind to a complementary base according to Watson-Crick base pairing can be used. By way of example only, by incorporating the adenine analog, 2,6-di-aminopurine (“2,6 AP”) instead of adenine in a nucleic acid sequence, and then incorporating that sequence into a fractal array receptor aggregate, the background Raman spectrum does not have the characteristic band at 733 cm⁻¹.

However, 2, 6 AP does not interfere substantially with its pairing with thymine (Hacia et al., Enhanced High Density Oligonucleotide Array-Based Sequence Analysis Using Modified Nucleoside Triphosphates Nucleic Acids Research 26:4975-4982 (1998), incorporated herein fully by reference). Subsequent binding of native nucleic acids that contain adenine cause the appearance of the Raman band at 733 cm⁻¹, and thus, the hybridization signal is specific for the native nucleotide sequence that binds to the receptor.

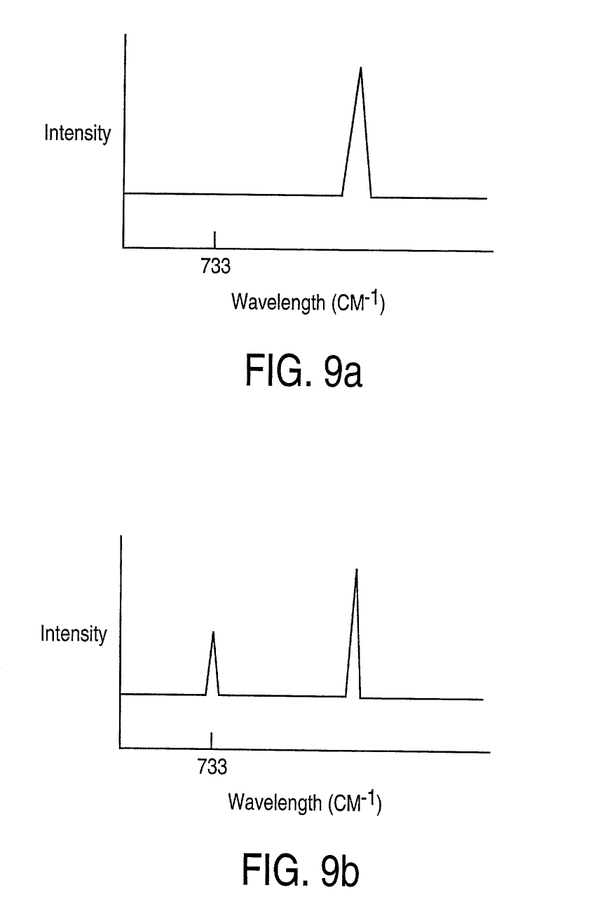

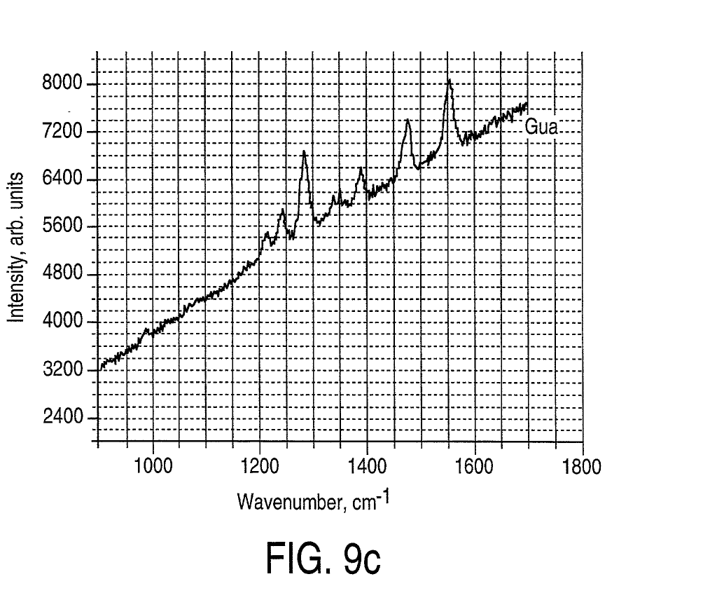

FIGS. 9a-9b are graphs illustrating the principle of use of an oligonucleotide receptor not having adenine in Raman spectroscopic detection of oligonucleic acids that contain adenine. FIG. 9a depicts a portion of a Raman spectrum of a nucleic acid not having adenine residues or other moieties having a Raman band at 733 cm⁻¹. FIG. 9b depicts the Raman spectrum obtained upon binding of an oligonucleotide containing adenine to a receptor molecule not having adenine as in FIG. 9a, showing the presence of a Raman band at 733 cm⁻¹.

### 2. Substitution of Thymine

In a fashion similar to that described above for adenine, RRS can be carried out using receptors in which thymine is replaced with any analog which lacks a characteristic Raman band and can form complementary base pairing with a nucleic acid according to Watson-Crick base pairing. By way of example only, thymidine can be replaced by 5-methyluridine in DNA oligomers attached to the matrix, without losing the capacity to hybridize with complementary bases (Hacia et al., Nucleic Acids Research 26:4975-4982 (1998), incorporated herein fully by reference). Thymine can also be replaced with 5-(1-propynyl)uridine.

### 3. Substitution of Guanine

Guanine has a hydrogen atom at position 8 that can be essentially completely replaced (by about 97%) with deuterium. This deuterium substitution can be carried out by incubation of the nucleic acid in D₂O at 90° C. according to the methods of Manor et al. An Isotope Edited Classical Raman Difference Spectoscopic Study of the Interactions of Guanine Nucleotides with Elongation Factor Tu and H-Ras p21, Biochemistry 30:10914-10920 (1991), incorporated herein fully by reference. Deuteration of guanine shifts the Raman band from about 1486 cm⁻¹ to about 1463 cm⁻¹ (Manor et al., Biochemistry 30:10914-10920 (1991), incorporated herein fully by reference.

The substitution of hydrogen by deuterium permits the manufacture of a RRS receptor oligonucleotide that is lacking in native guanine. Therefore, upon hybridization to the deuterated guanine receptor, the native guanine provides the characteristic Raman band indicating the presence of the analyte bound to the receptor oligonucleotide.

FIG. 9c depicts a graph of wavenumber in cm⁻¹ and the intensity of Raman scattering observed (in arbitrary units) of a sample containing 500 mg/ml guanine in 10 M KOH. Excitation is at 514 nm with a laser power of 1 Watt and the spectrum was acquired within 3 seconds using a charge coupled device (CCD). Peaks in intensity can be observed at about 980 cm⁻¹, about 1220 cm⁻¹, about 1240 cm⁻¹, about 1280 cm⁻, 1345 cm⁻¹, about 1390 cm⁻¹, about 1470 cm⁻¹ and about 1555 cm⁻¹. This pattern of peaks is similar to the pattern previously published. Without enhancement, this system can detect guanine in a concentration of about 20 mg/ml.

### 4. Peptide Nucleic Acids

As described above for substitution of adenine, thymine and guanine in oligonucleic acid receptors, replacement of those bases in peptide nucleic acids can also result in the manufacture of receptors that lack a characteristic Raman band of a native nucleic acid. Upon binding the native analyte, the characteristic Raman band can be detected and quantified.

B. Modified Protein Receptors

In fashions analogous to those described above for modified nucleic acid receptors, according to the methods of this invention, one can synthesize protein receptors that lack a characteristic signal found in native proteins. By way of example only, the amino acid, tryptophan (“Trp”), in an antibody can be replaced by z⁷Trp during the synthesis of the antibody. Z⁷Trp is disclosed in Cornish et al., Site-specific Incorporation of Biophysical Probes Into Proteins, Proceedings of the National Academy of Science (USA): 91:2910-2914 (1994), herein incorporated fully by reference. Alternatively, antibodies directed toward insulin or other cysteine-containing proteins and peptides can be prepared without cysteines, the cysteines being replaced with selenocysteine or homocysteine. The Raman spectral features associated with cysteine or cystine can be clearly detected in the presence of such antibody receptors. Moreover, other artificial amino acids can be used to replace native amino acids in proteins. Any artificial amino acid that lacks a characteristic Raman signal and does not substantially disrupt secondary, tertiary or quaternary structures be advantageously used in RSS. Thus, using RRS, a native protein binding to a substituted receptor can be used to detect the protein analyte. Similarly, deuterium (D) can replace hydrogen in certain proteins.

C. Detection Using Unmodified Receptors

In certain other embodiments of this invention, it is not necessary to substitute an analog that lacks a characteristic Raman band into a receptor molecule. Hybridization of complementary oligomeric nucleic acids can shift the Raman spectrum. However, the magnitude of the shifts caused by hybridization are relatively small, so it can be desirable to enhance specific Raman features to increase the signal.

D. Specificity of Ligand-Receptor Binding

The level of specificity of an assay of this invention can depend on the purposes of the assay. For example, if the aim of the assay is the detection of any of a series of related nucleotide sequences, herein termed “homologues,” the fidelity of the hybridization reaction need not be as high as an assay in which the detection and identification of single nucleotide polymorphisms (“SNPs”). The methods and compositions of this invention are well suited to detecting the presence or absence of a Raman band within a particular cell of a matrix array. Moreover, because the intensity of a characteristic Raman band is increased as the number of bound analyte moieties increases, the methods of this invention can be used to quantify the amounts of analytes in an assay.

In general, the specificity of nucleotide-nucleotide hybridization reactions can depend on the conditions of hybridizations, herein termed “stringency.” Hybridization conditions are described in Sambrook et al., Molecular Cloning: A Laboratory Manual, Second Edition, Cold Springs Harbor Laboratory Press (1989), incorporated herein fully by reference. In general, as used herein, the term “high stringency” refers to conditions in which the temperature of hybridization is about 5° C. to about 10° C. below the melting temperature of the duplex. The melting temperature_{™} of an oligonucleotide duplex can be estimated as follows:

Tₘ=81.5−16.6(log₁₀[Na⁺])+0.41(fraction C+G)−(600/N),

where [Na⁺] is the sodium concentration, C+G is the amount of cytosine (C) and guanine (G) as a fraction of the total number of nucleotide bases, and N is the chain length. High stringency involves either the incubation of or the washing of ligand and receptor nucleotides under conditions that disfavor hybridization of non-complementary sequences. Such conditions include the use of high temperatures, low salt concentration and high detergent concentrations. Using high stringency, detection of sequences having only one non-complementary base (one “mismatch”) can be accomplished. Conversely, low stringency conditions include lower temperatures, higher salt concentrations and lower concentrations of detergents. Low stringency conditions can be especially desirable if the purpose of the assay is the detection of homologues, in which base-pair mismatches are present.

Moreover, in certain embodiments of this invention, one can obtain qualitative information regarding the number of base-pair mismatches by making repeated Raman spectroscopic measurements of the same cell under progressively higher stringency conditions. For example, if an analyte has a relatively large number of mismatches, so that a detectable Raman signal is present only after low stringency washing, subsequent washing of the same cell at high stringency conditions can remove the analyte from that cell. This stringency is herein termed the “stringency threshold.” By comparing the number of mismatches with the stringency threshold, one can determine the relative degrees of homology of nucleic acid sequences without determining the actual sequences.

The specificity of binding of analytes is often not perfect, especially when antibodies are used. Antibodies can bind other analytes non-specifically, in addition to their direct targets. In such situations, spectral analysis of Raman spectral features can permit discrimination and quantitation of the desired analyte even in the presence of non-specific binding.

E. Detection of Analytes By Raman Spectroscopy

### 1. Raman Spectroscopy of Analytes

Devices used to perform analyses according to the methods of this invention can include any device that can produce laser light of the wavelengths needed for analysis. For example, the T64000 Raman Spectrometer (The Ultimate Raman Spectrometer Instruments S.A. Ltd. (UK) can be used. Alternatively, one can use a Raman System 2000 produced by Chromex, Inc. Albuquerque, New Mexico, or other suitable Raman devices. Desirable features of a suitable instrument include the ability to position the sample compartment to adjust the sensitivity of the spectrum, provides for low frequency measurements, provides adequate spectral resolution, and a liquid nitrogen cooled charged coupled device (“CCD”) detector. The spectrometer is suitably equipped with a laser light source comprising a continuous wave, frequency doubled argon laser. Because the purine and pyrimidine ring structures of nucleotides have characteristic absorption maxima in the ultraviolet range, it can be desirable to provide laser light having emission wavelengths in the ultraviolet range. A suitable laser is the Inova 300 FReD, available from Coherent Inc., Santa Clara, Calif. Laser power for certain embodiments of this invention can be maintained at about 5 milliwatts at 257 nm, or 1 milliWatt at 244 nm, 229 nm and 238 nm.

For other applications, it can be desirable to use wavelengths in the range of about 200 nm to about 2000 nm. In certain of these embodiments, it can be desirable to use wavelengths in the range of about 1000 to 1100 nm, alternatively at 1064 nm (Nd-YAG laser). Long wavelengths, for example from about 780 nm to about 2000 can be desirable to avoid fluorescence. Wavelengths below about 250 nm can also be desirable to avoid fluorescence. Such lasers having wavelengths below about 250 are commercially available.

For other applications, it can be desirable to use longer wavelengths, for example, in the range of about 830 nm. Such a light source is a continuous-wave titanium:sapphire laser. One commercially available Raman spectrometer system (Raman System 2000) operates at about 785 nm. For other applications, wavelengths in the visible range can be suitable.

To detect analytes in a single cell, it can be desirable to provide Raman spectroscopic measurements over areas that are sufficiently small to avoid cross-readings from adjacent cells. For matrix arrays having 100 μm×100 μm per side, it is desirable to provide a narrow, focused beam of incident light.

IV. Analysis of Data

To determine whether a cell has bound analyte, all that is needed is to compare the intensity of the characteristic Raman bands of a cell before exposure to the mixture of analytes to the intensity of the same Raman bands in the same cell after exposure to the analyte. Alternatively, for matrix arrays in which the receptors all have a characteristic Substituent, (e.g., 2,6 AP), one can use any cell prior to analyte exposure as a reference cell.

The reference cell can typically exhibit a Raman spectrum having several bands corresponding to invariant molecules. Such can be an internal standard for the comparison of cells having bound analyte. Moreover, if desired, one can incorporate into each cell, a known reference Raman label that is not present in the analyte sample. Thus, upon exposure of the cell to light under conditions of analysis, any change in light transmission or absorption that is due to non-specific Raman scattering can be evaluated in situ.

For determination of whether analyte-receptor binding occurs, a threshold increase in the intensity of a Raman spectral feature can be selected. For measurements not requiring quantification of analyte-receptor binding, this threshold can be set to a convenient, high level. For example, about 25% of the maximal signal. For alternative embodiments, in which the intensity of Raman signal is to be carefully assessed, it can be desirable to set the threshold to a lower value, for example, 2-5% of the maximal Raman signal.

Once the presence and/or amount of analyte is determined, subsequent operations can be carried out to provide additional information. For example, if the analysis is to determine the presence of an oligonucleotide having a desired sequence, the intensity of Raman signal from related cells can be compared. If a series of cells contains receptors having overlapping oligonucleotide sequences, as described, for example, in U.S. Pat. No. 5,925,525, incorporated herein fully by reference, then the presence of analyte in the related cells can provide information concerning the sequence and overall size of the particular analyte in question.

### EXAMPLE 6

### Manufacture of Nested Particle Associates

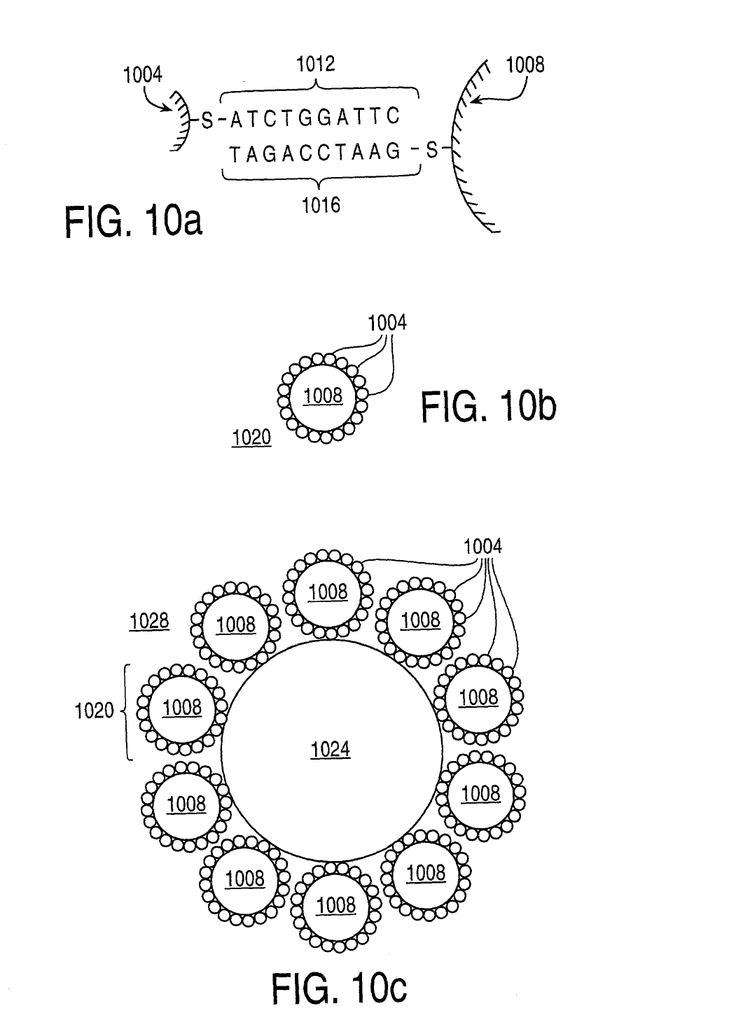

By way of example only, a nested particle associates can be made by selecting colloidal solutions of metal gold particles of uniform size, being 10 nm, 40 nm and 240 nm in diameter, respectively. FIGS. 10a-10c depict the manufacture of a nested particle structure made from such particles.

FIG. 10a depicts a 10 nm gold particle 1004 having a DNA linker 1012 attached thereto. 40 nm particle 1008 having DNA linker 1016 being complementary to DNA linker 1012 is attached to particle 1008. Mixtures of particles 1004 and 1008 are placed in solution and interact with each other DNA linkers to form a first-order nested structure 1020 as shown in FIG. 10b.

FIG. 10c depicts a second-order nested particle structure having particles 1004 and 1008 as shown in FIGS. 10a and 10b, but with the addition of a larger particle 1024 having a diameter of 240 nm, surrounded by first order nested particles 1020 to form second order nested particle 1028. Heating the mixture of first-order or second-order to a temperature less than about 100° C. and then cooling the mixture can result in better ordering of the nested particles.

### EXAMPLE 7

### Manufacture of Surfaces Having Non-Random Particle Structures

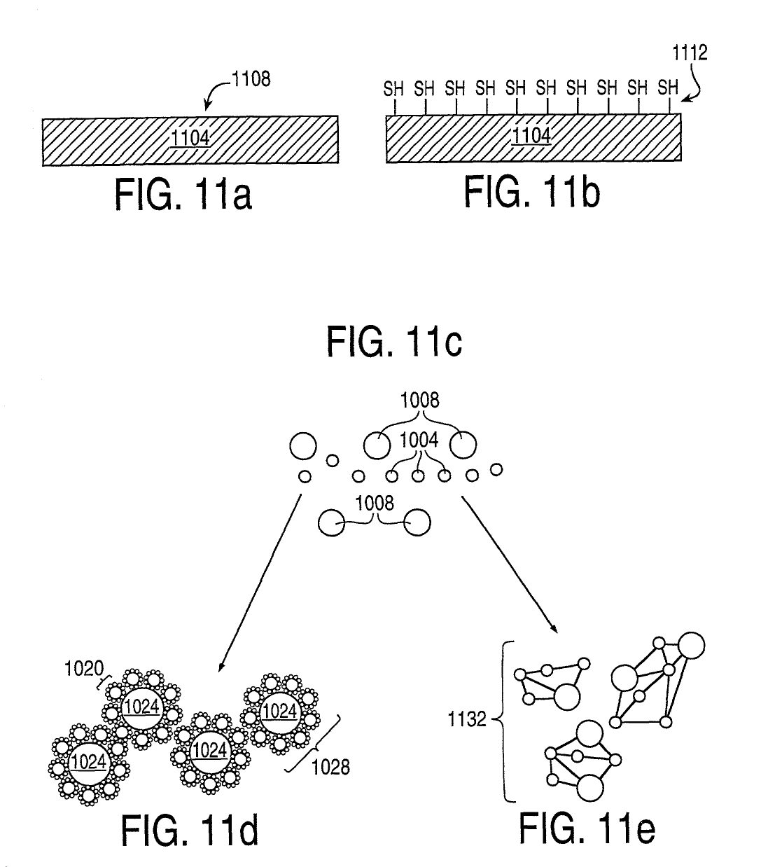

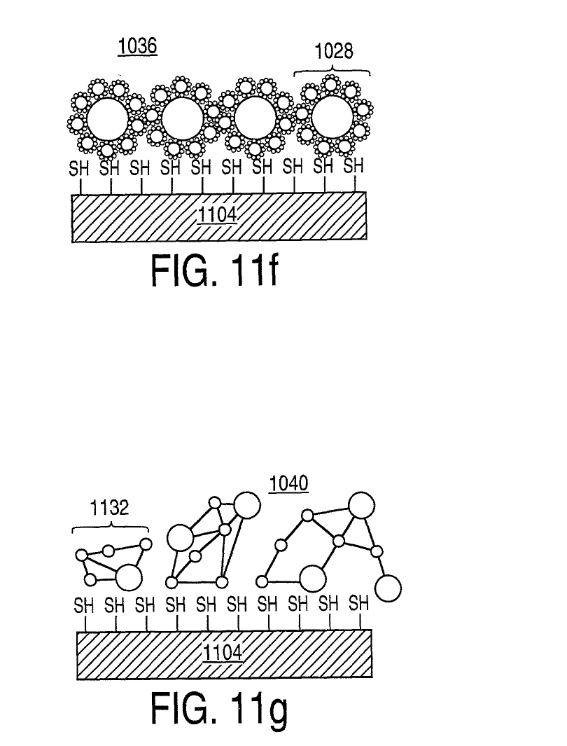

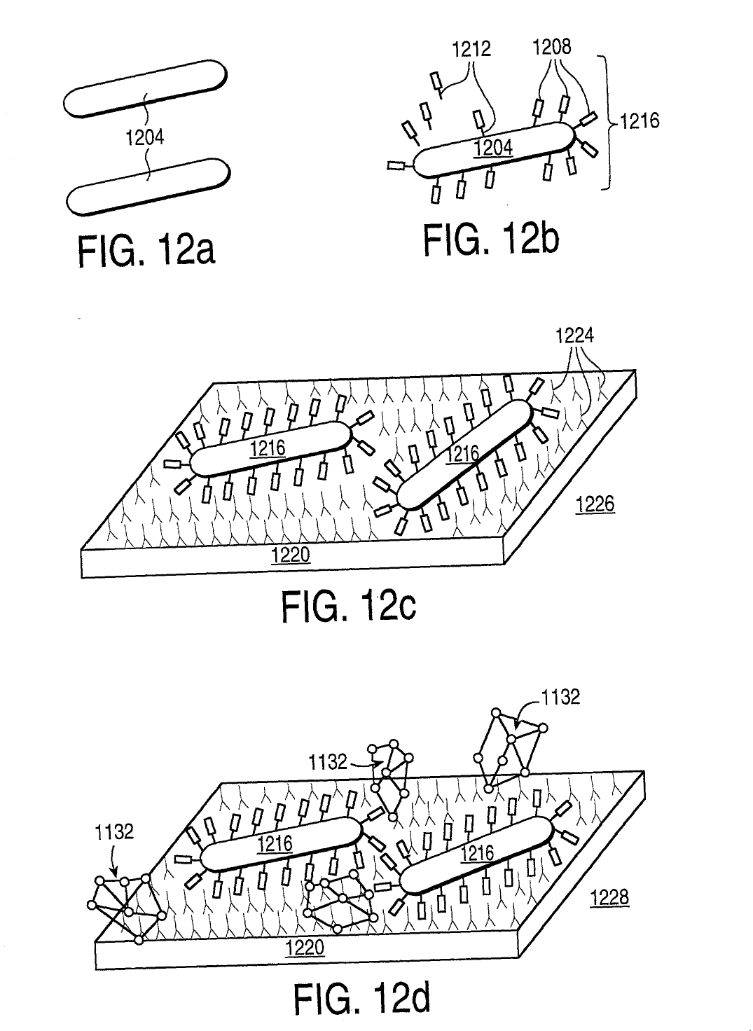

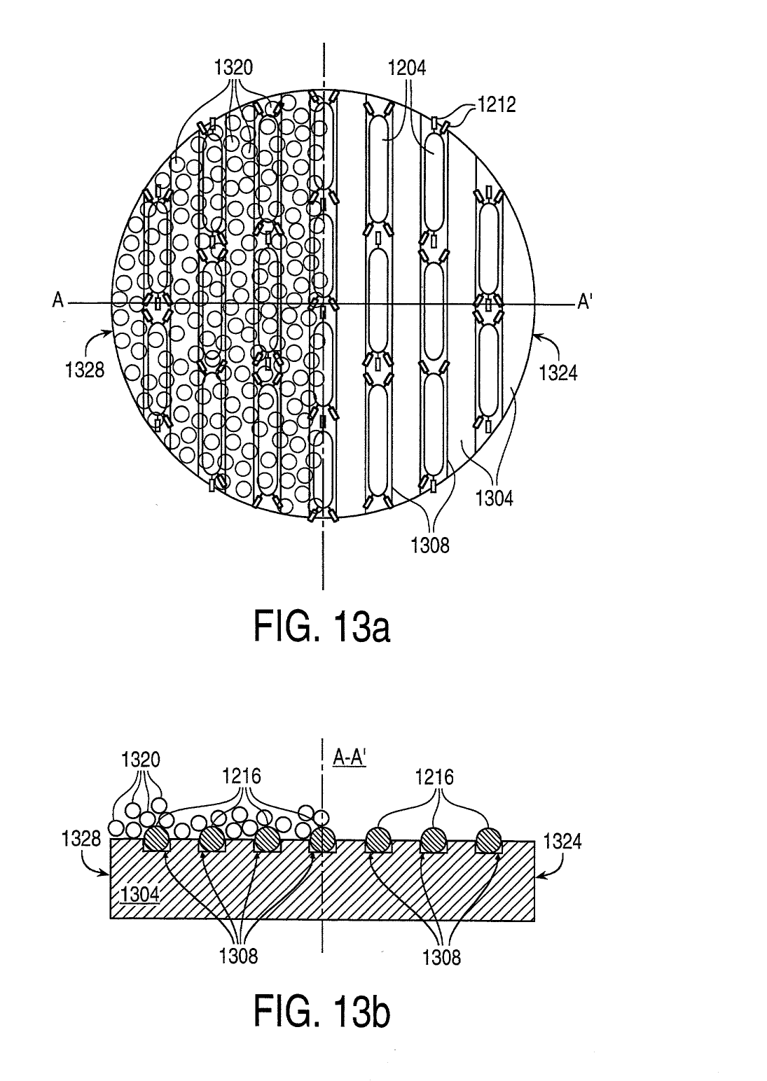

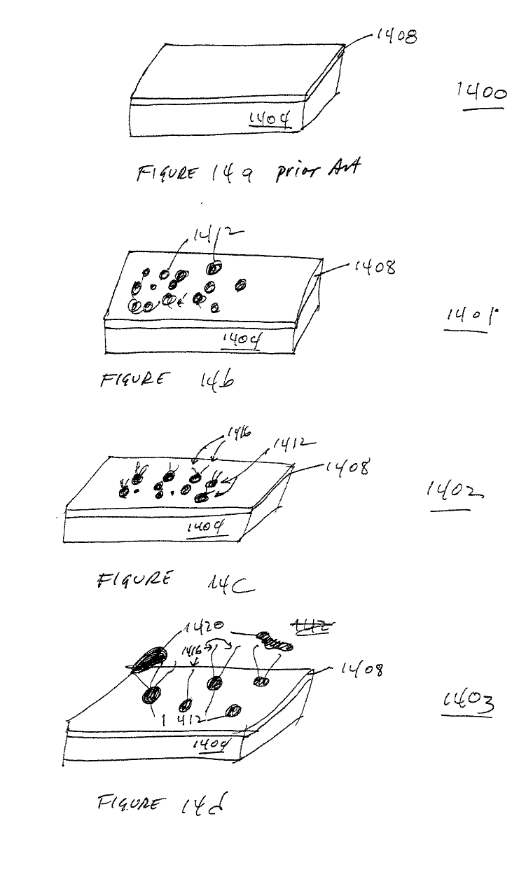

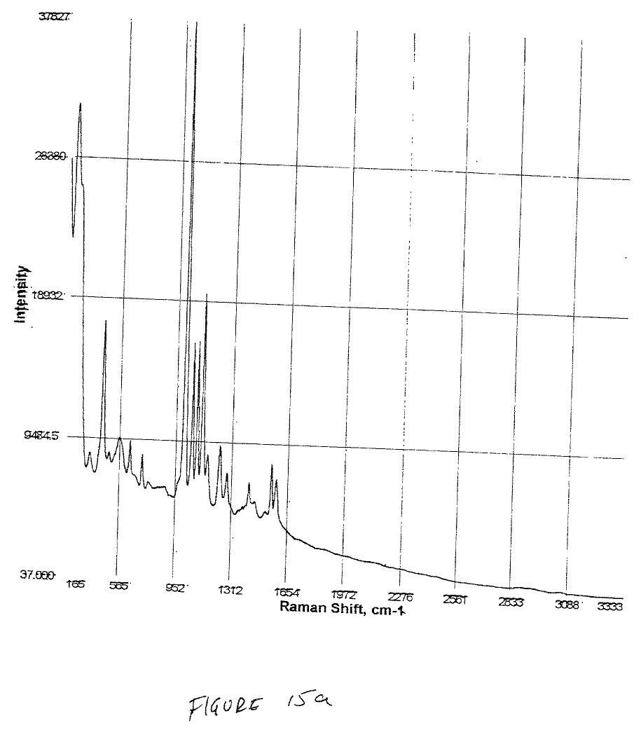

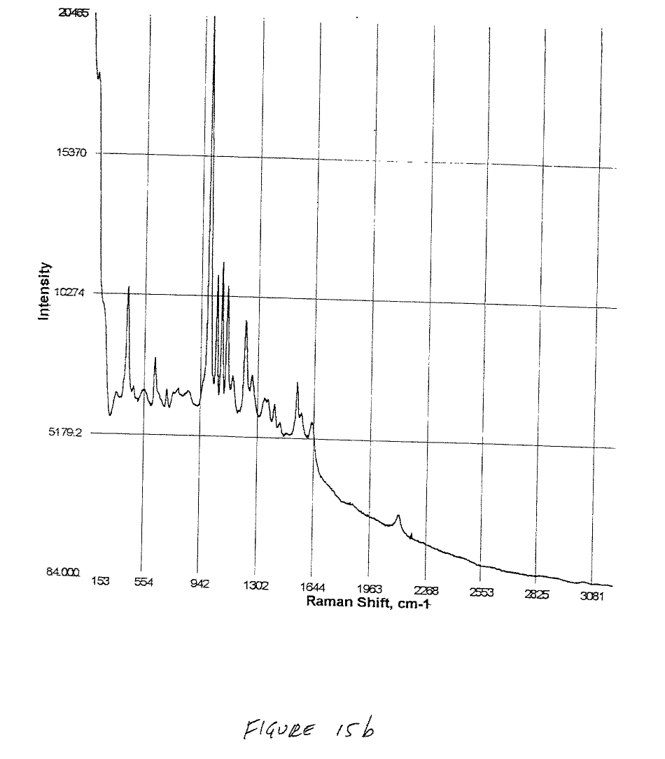

FIGS. 11a-11g depict alternative embodiments of surfaces having fractal particle structures thereon. FIG. 11a depicts a substrate 1104 having a top surface 1108. FIG. 11b depicts the surface 1008 as shown in FIG. 1 a after being activated, resulting in thiol groups 1112 attached to surface 1108.

FIG. 11c depicts a plurality of particles 1004 being smaller than intermediate particles 1008. FIG. 11d depicts second-order nested particle structures 1028 made from first-order nested particles structures 1020 made from the small particles 1004, the intermediate particles 1008 and larger particles 1024.

FIG. 11e depicts chemically linked particle structures 1132 made from small particles 1004 and intermediate particles 1008.

FIG. 11f depicts an electromagnetic signal enhancer 1132 having substrate 1104 with nested particle structures 1028 thereon.

FIG. 11g depicts an alternative electromagnetic signal enhancer 1040 comprising substrate 1104 with linked particle structures 1132 as shown in FIG. 11e thereon.

### EXAMPLE 8

### Manufacture of Biochips with Analyte Receptors and Fractal Particle Structures

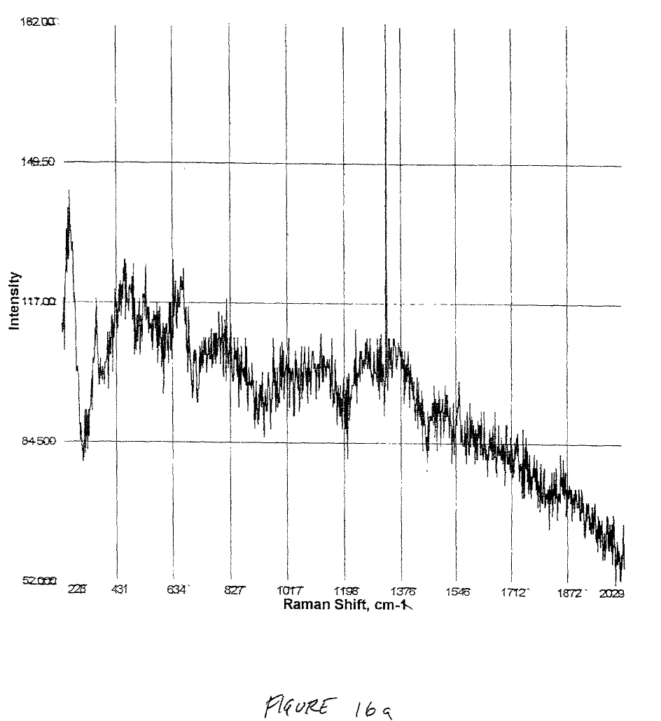

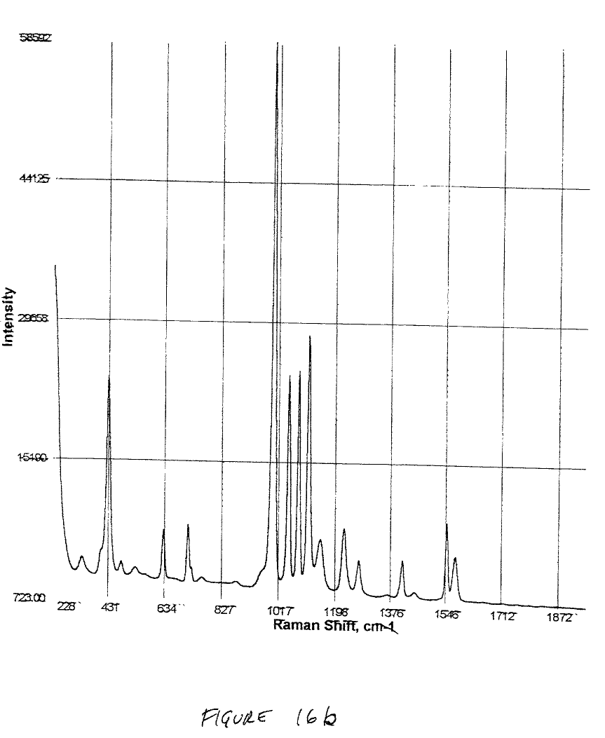

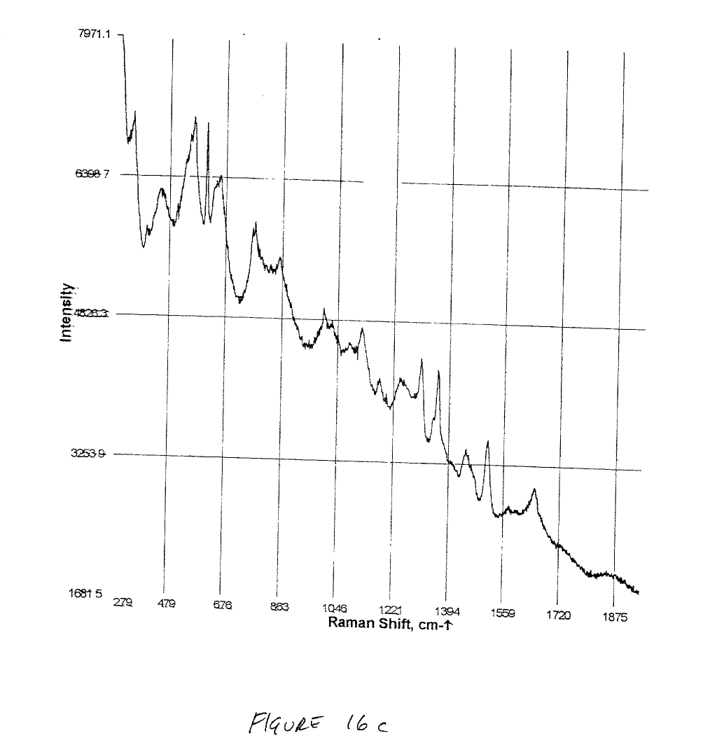

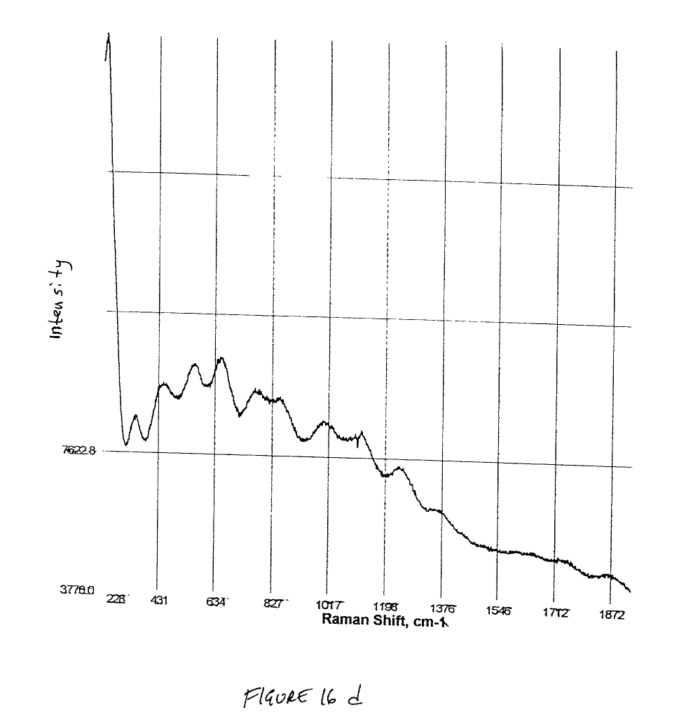

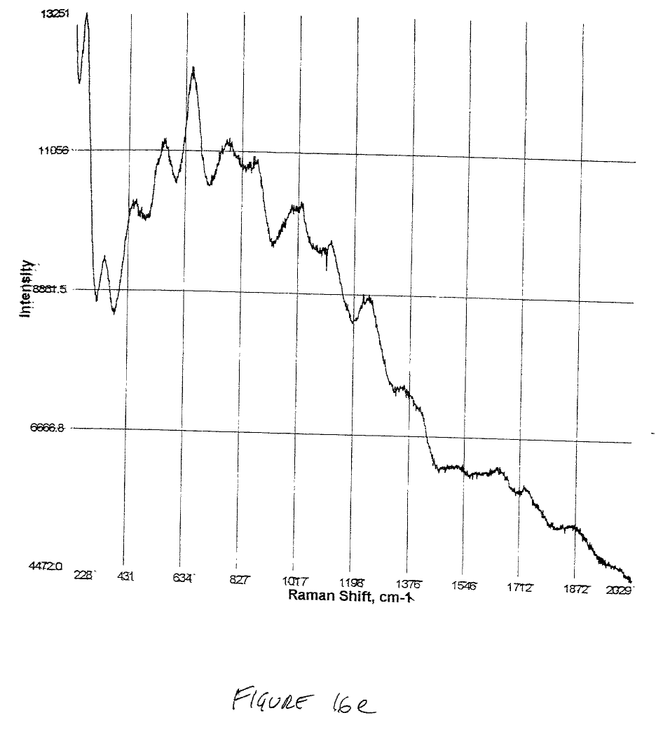

FIGS. 12a-12d depict the manufacture of a biochip having analyte receptors and enhancers. FIG. 12a depicts two rod-shaped particles 1204. FIG. 12b depicts the rod-shaped particles shown in FIG. 12a and analyte receptors 1208 with connectors 1212. Some of the analyte receptors 1208 are shown attached to rod 1024 by connectors 1212 forming receptor-rod complex 1216.

FIG. 12c depicts a biochip 1226 comprised of substrate 1220 with linkers 1224 and having receptor-rod complexes 1216 attached thereto.

FIG. 12d depicts an alternative biochip 1228, similar to biochip 1216 depicted in FIG. 12c, but further comprising linked particle structures 1132 as depicted in FIG. 11e.

### EXAMPLE 9

### Biochip Made With Non-Nested Particles

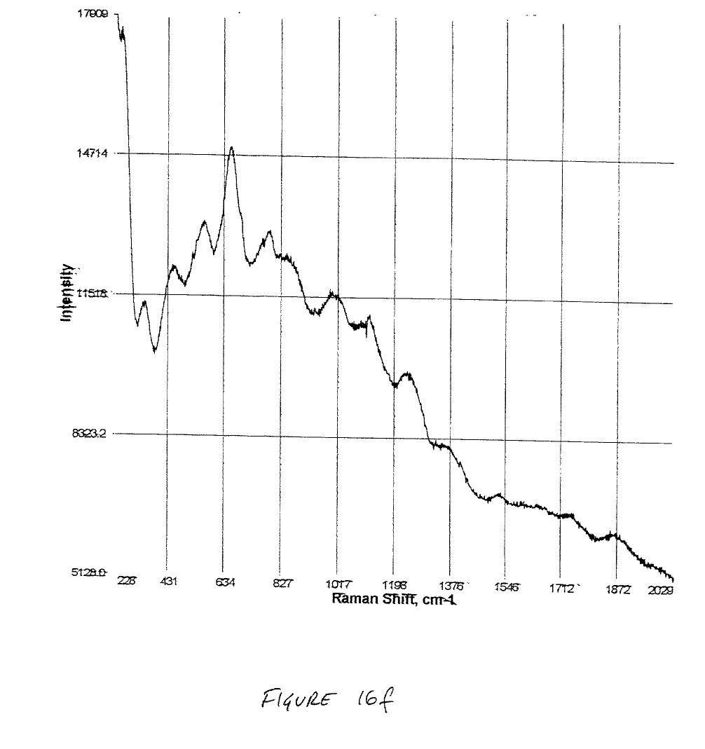

FIGS. 13a and 13b depict two views of additional embodiments 1324 of this invention having receptor-rod complexes and non-nested particles.

FIG. 13a depicts a top view of a biochip having two types of structures. On the right side of FIG. 13a, structure 1324 has linearly arranged rods 1204 having receptors 1212 attached thereto as depicted in FIG. 12b. The rods 1204 are depicted as being present within trenches 1308. Some rods 1204 are shown parallel to each other, and others are shown end-to-end, although other configurations are within the scope of this invention. The right side of FIG. 13b depicts a cross-sectional view along line A-A′ through the embodiment 1324 depicted on the right side of FIG. 13a. Trenches 1308 have receptor-rod complexes 1216 therein. The trenches 1308 can be either parallel as shown, or can be non-parallel.

The left side of FIG. 13a depicts an alternative biochip, comprising the biochip as depicted in embodiment 1324 but additionally having particles 1320 distributed over the substrate 1304 and the receptor-rod complexes. Particles 1320 can be made, for example, by laser ablation.

The left side of FIG. 13b depicts a cross-sectional view along line A-A′ of the embodiment 1328 as shown in FIG. 13a. Substrate 1304 has trenches 1308 with receptor-rod complexes 1216 therein, and having particles 1320 over the top of the substrate 1304 and receptor-rod complexes 1216.

### EXAMPLE 10

### Preparation of Fractal Silver Aggregates using Citrate

Upon dissolving 45 mg AgNO₃ (Sigma) in 250 ml triple distilled, deionized water, the solution was brought to boiling under stirring with magnetic stirrer. A solution of 1% sodium citrate (5 ml) was added drop-by-drop to the boiling solution under vigorous stirring. The solution was kept boiling for 60 min, while a tap water cooled condenser prevented loss of water due to evaporation. The resulting solution of colloidal silver was kept in a dark glass bottle at 5°-8° C. for a period of up to at least several months.

Fractal aggregates of silver particles were produced by mixing 250 microliters of the colloidal silver solution with 1750 microliters of a water solution of NaCl (Sigma, ultra pure) to bring the final concentration of NaCl to 60 mM. The aggregates formed within several minutes and the solution was fairly stable for at least about half an hour. Fractal aggregates were applied to a metal coated substrate, and the fractal aggregates sedimented and adhered to the metal surface, forming a fractal associate-derivatized metal surface.

### EXAMPLE 11

### Preparation of Fractal Silver Aggregates using NaBH₄

A solution of 5 mM AgNO₃ (Sigma) in triple distilled, deionized water (50 ml) was added drop-by-drop to 150 ml ice-cold, 2 mM solution NaBH₄ (Sigma), under vigorous stirring with magnetic stirrer. Right after that, a solution of 1% poly(vinyl alcohol) (25 ml) was added. The mixture was then is boiled for 60 min, while a tap water-cooled condenser prevented loss of water due to evaporation. The solution of colloidal silver was kept in a dark glass bottle at 5°-8° C. for a period of up to at least several weeks.

Fractal aggregates of silver particles were produced by mixing 250 microliters of the colloidal silver solution with 1750 microliters of a water solution of NaCl (Sigma, ultra pure) to bring the final concentration of NaCl to about 100 mM. The aggregates formed within several minutes and the solution was fairly stable for at least up to about half an hour. Fractal aggregates were applied to a metal coated substrate, and the fractal aggregates sedimented and adhered to the metal surface, forming a fractal associate-derivatized metal surface.

### EXAMPLE 12

### Preparation of Fractal Gold Aggregates Using Citrate

Upon dissolving 120 mg HAuCl₄ (Sigma) in 250 ml triple distilled, deionized water, the solution was brought to boiling under stirring with magnetic stirrer. A solution of 1% sodium citrate (5 ml) was added drop-by-drop to the boiling solution under vigorous stirring. The solution was kept boiling for 60 min, while a tap water-cooled condenser prevented loss of water due to evaporation. The solution of colloidal gold was kept in a dark glass bottle at a temperature of about 5°-8° C. for up to at least about several weeks.

Fractal aggregates of gold particles were produced by mixing 250 microliters of the colloidal gold solution with 1750 microliters of a water solution of NaCl (Sigma, ultra pure) to bring the final concentration of NaCl to ca. 100 mM. The aggregates formed within several minutes and the solution is stable for at least about half an hour. Fractal aggregates were applied to a metal coated substrate, and the fractal aggregates sedimented and adhered to the metal surface, forming a fractal associate-derivatized metal surface.

In other embodiments, substrates can be prepared having a metal surface, such as gold, and also having fractal aggregates made of silver colloid as described above in Examples 10 and 11 or alternatively having gold fractal aggregates as in Example 12.

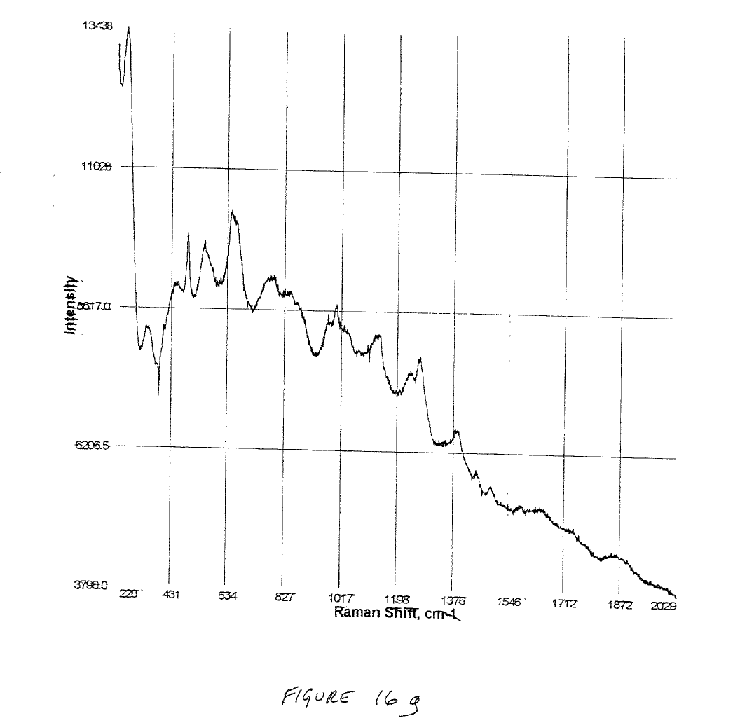

FIGS. 14a-14d depict embodiments of this invention. FIG. 14a depicts a prior art slide 1400 in which a substrate 1404 has a layer of gold metal 1408 thereon. FIG. 14b depicts an embodiment 1401 of this invention in which the substrate 1404 has a layer of gold 1408 thereon and has silver or gold fractal aggregates 1412 thereon. FIG. 14c depicts an embodiment 1402 of this invention, in which fractal aggregates 1412 have receptors 1416 thereon. FIG. 14d depicts an embodiment of this invention 1403, similar to those embodiments shown in FIGS. 14b and 14c, and having analytes 1420 in proximity to receptors 1416.

### EXAMPLE 13

### Enhancement of Raman Spectra of DTP on Fractal-Derivatized Substrates

In this series of experiments, we determined that fractal-derivatized substrates can be used to enhance Raman signals from analytes. We selected a silver fractal-derivatized gold substrate as described above for Example 10. To this substrate, we added a solution comprising 2,2′-dithio-dipyridine (DTP; Sigma Chemical). 20 mg of DTP was added to 1 ml of water (triple distilled deionized) and shaken for 1 minute in a shaker. We permitted the undissolved DTP to settle out of solution and applied 10 microliters (μl) of this solution to 1 ml of water. Raman spectrum of this diluted solution showed no detectible signal characteristic of DTP.

Then, we added 25 microliters of this solution to a fractal-derivatized substrate and permitted the DTP to adhere to the substrate. We then measured the Raman signal and found a pattern of Raman bands characteristic of DTP.

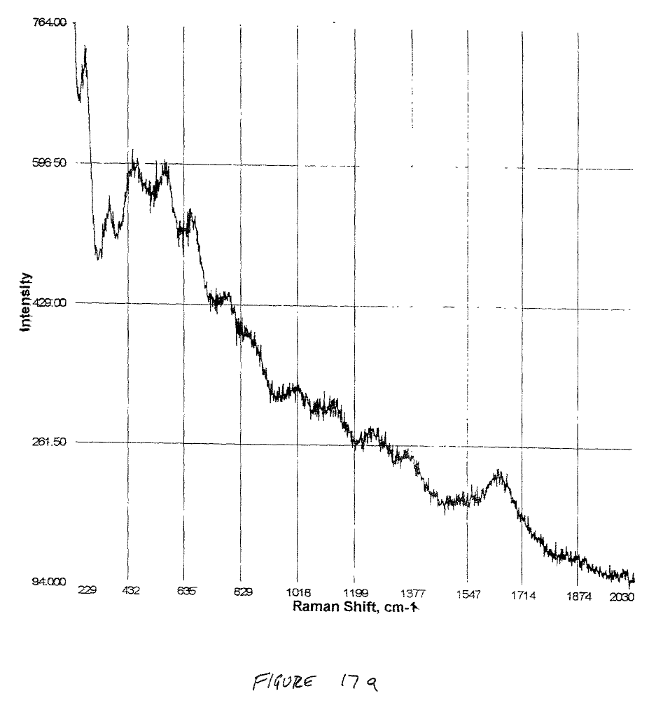

FIG. 15a depicts the Raman spectrum of DTP on a gold-coated substrate having silver fractal structures thereon. Several distinct peaks characteristic of DTP were observed. FIG. 15b depicts the Raman spectrum of DTP on a gold-coated substrate having gold fractal structures thereon. Again, we found a series of distinct peaks characteristic of DTP as seen in FIG. 15a. We conclude from these experiments that devices of this invention incorporating silver and gold fractal aggregates can be used to enhance Raman signals generated by DTP.

Thus these studies demonstrate (1) that silver and gold fractal-derivatized substrates each can effectively enhance Raman signals, and (2) that such surfaces can be effectively cleaned. Moreover, using these methods, one can control the effectiveness of formation of fractal structures, the attachment of those structures to a substrate, the attachment of a Raman signal generator to fractal structures and the removal of Raman signal generators from substrates. These tools can be widely applied to a wide variety of fractal aggregates, substrates, receptors and analytes. Moreover, DTP can be used as an effective probe of the quality of the fractal aggregates used to enhance spectrographic analysis of analytes.

### EXAMPLE 14

### Fractal-Derivatized Substrate Having Reduced Glutathione Receptors for Glutathione S-Transferase

In this series of experiments we wanted to prepare a fractal-derivatized substrate having a receptor attached thereon to be used to bind and detect an analyte using Raman spectroscopy. We used reduced glutathione as a receptor and glutathione S-transferase (“GST”) as an analyte. Glutathione is a tripeptide consisting of the three amino acids in order: Glycine-Cysteine-Glutamic acid (Gly-Cys-Glu). It is known in the art that glutathione S-transferase binds to reduced glutathione. Thus, reduced glutathione can be a receptor for binding and analysis of glutathione S-transferase.

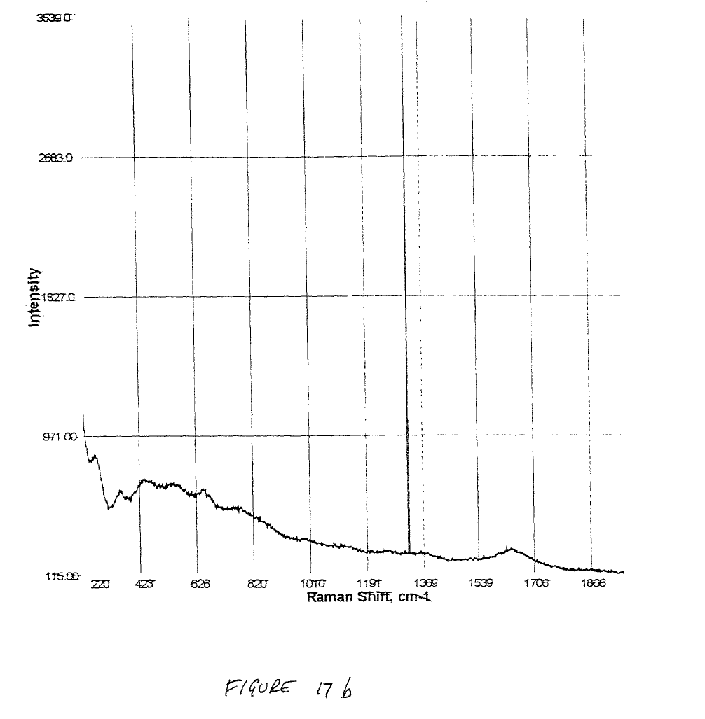

To manufacture a fractal-derivatized substrate, we obtained a quartz substrate having a preformed layer of gold metal thereon. Such slides are commercially available from EMF Corporation, Ithaca, N.Y. The slides were washed for one (1) hour in concentrated nitric acid (HNO₃) to clean the slides, then extensively rinsed with a large excess of distilled deionized water and air dried. FIG. 16a depicts a Raman spectrum of a commercially available gold-coated quartz slide that had been washed with HNO₃ and water as described above. FIG. 16a shows only very weak Raman signals, with no discernable pattern within the noise. Such spectra are characteristic of clean, gold-covered quartz slides.

Then, we prepared silver fractal aggregates as described above in Example 10. To test the ability of the fractal aggregates to enhance Raman signals, we exposed a small sample of fractal aggregates to DTP.

FIG. 16b depicts Raman spectrum of such a preparation of saturated DTP diluted 100 fold in the presence of silver fractal aggregates prepared using the citrate process as described above in Example 10. A pattern of sharp Raman peaks characteristic of DTP indicates that the fractal aggregates substantially enhanced the Raman signals produced by the DTP. This study demonstrated that the fractal aggregates used to coat gold-covered slides were effective in enhancing Raman signals.

To derivatize the slides with fractal structures, we covered the portion of the slide to be so derivatized with solution of silver colloid and NaCl at a concentration of about 60 mM to about 120 mM. We then permitted the silver fractal aggregates to adhere to the gold surface. FIG. 16c depicts the Raman spectrum derived from a gold-covered slide having silver fractal aggregates thereon without any receptor. We then rinsed the fractal-derivatized substrate overnight with saturated solution of NaCl in water, followed by rinsing with triple distilled-deionized water to wash away non-adherent fractal aggregates. The slide was then air dried.

FIG. 16d depicts Raman spectrum of a slide washed in the above described fashion. The graph shows small changes in intensity that are unrelated to the presence of a receptor or an analyte. The pattern is characteristic of slides produced in this fashion, and is reproducible between slides. This process removed remaining unbound fractal aggregates and the reagents and contaminants present in the colloid.

The resulting fractal-derivatized substrate was used for the attachment of a receptor molecule, reduced glutathione. A solution of reduced glutathione was prepared by dissolving commercially available reduced glutathione (Sigma Chemical Co., St. Louis Mo.) in triple distilled-deionized water to a final concentration of 100 mg/ml. The solution of reduced glutathione was applied to the fractal-derivatized substrate and was permitted to adhere for a period of 30 minutes at room temperature (about 22° C.). After that time, the substrate was rinsed with water to remove unbound reduced glutathione. After washing, the substrate was air dried for 10 minutes.

At this point, a Raman spectrum of the substrate was obtained, and showed the characteristic pattern of reduced glutathione. FIG. 16e depicts a Raman spectrum of reduced glutathione attached to a silver fractal-derivatized gold-coated substrate. A sharp peak in Raman signal was observed at about 650 cm⁻¹, characteristic of reduced glutathione. This experiment demonstrated that reduced glutathione (a “receptor”) was attached to a surface having fractal aggregates sufficiently near to enhance Raman signals from the reduced glutathione.

Subsequently, we extensively rinsed the glutathione-coated slide with triple-distilled, deionized water. FIG. 16f depicts the Raman spectrum obtained after rinsing and drying the slide. The characteristic Raman peak of reduced glutathione at about 650 cm⁻¹ is present, and has about the same magnitude as in FIG. 16e. From this study, we conclude that the receptor (glutathione) is firmly attached to the substrate, and is sufficiently close to the fractal aggregates to substantially enhance the Raman signals generated by glutathione.

After preparation as described above, the substrate was exposed to a solution comprising glutathione S-transferase (Sigma Chemical Co., St. Louis, Mo.; “GST”) at a concentration of 20 mg/ml in a buffer comprising 10 mM HEPES (Sigma Chemical Co.), 100 mM NaCl, pH 7.0. The GST was permitted to adhere to the reduced glutathione for a period of 5 minutes and then the Raman spectrum was acquired.

FIG. 16g depicts the Raman spectrum obtained after addition of GST. The Raman peak characteristic of glutathione is present at about 650 cm⁻¹. In addition, several other peaks are present. These peaks can be used to detect the presence of GST.

We found that in the absence of enhancing structures, GST alone was not detected. In the presence of fractal enhancing structures, however, glutathione S-transferase did results in a detectable Raman signal. Moreover, in the presence of both enhancing structures and glutathione, GST produced a Raman signal different from that of either glutathione alone or GST alone.

These results show that reduced glutathione can be detected in the presence of fractal aggregates. These results also show that a protein analyte, GST, can be detected, and that GST can be identified by Raman spectroscopy in the presence of reduced glutathione attached to fractal structures.

In other embodiments of this invention, polymers comprising glutathione can be provided near fractal aggregates. In certain of these embodiments, a polymeric structure can have glutathione side chains, attached by way of the glycine residues, or alternatively, by SH groups. In these embodiments, the polymer can have the appearance of a “bottle brush”, having a plurality of glutathione chains available for association with GST.

In other embodiments, a single Gly-Cys-Glu terminus can be provided, on a long chain polymer. In these embodiments, there need not be a plurality of glutathione moieties. These embodiments comprising bottle brush or linear reduced glutathione can provide the receptors with sufficiently long linkers to permit the association of the glutathione moiety and the binding site of GST.

### EXAMPLE 15

### Fractal-Derivatized Substrate Having Gly-Cys Receptors for Glutathione S-Transferase

We performed additional experiments as described above in Example 14 except that instead of using reduced glutathione, we used the dipeptide glutathione fragment, Gly-Cys. Slides having Gly-Cys were prepared as described above for reduced glutathione. We found that the Raman signal observed for Gly-Cys-glutathione S-transferase was different than for either Gly-Cys or glutathione S-transferase alone. These results indicate that glutathione S-transferase can be detected using Raman spectroscopy as part of a complex with Gly-Cys.

### EXAMPLE 16

### Fractal-Derivatized Substrate Having Cys-Glu Receptors for Glutathione S-Transferase

We performed additional experiments as described above in Example 14 except that instead of using reduced glutathione, we used the dipeptide glutathione fragment, Cys-Glu. Slides having Cys-Glu were prepared as described above for reduced glutathione. We found that the Raman signal observed for Cys-Glu-glutathione S-transferase was different than for either Cys-Glu or glutathione S-transferase alone. These results indicate that glutathione S-transferase can be detected using Raman spectroscopy as part of a complex with Cys-Glu.

### EXAMPLE 17

### Fractal-Derivatized Substrate Having Cysteine Receptors for Glutathione S-Transferase

We performed additional experiments as described above in Example 14 except that instead of using reduced glutathione, we used the amino acid Cysteine (Cys). Slides having Cys were prepared as described above for reduced glutathione. We found that the Raman signal observed for Cys-glutathione S-transferase was different than for either Cys or glutathione S-transferase alone. These results indicate that glutathione S-transferase can be detected using Raman spectroscopy as part of a complex with Cysteine.

### EXAMPLE 18

### Fractal Enhancement of Rhodamine Detection by Raman Spectroscopy

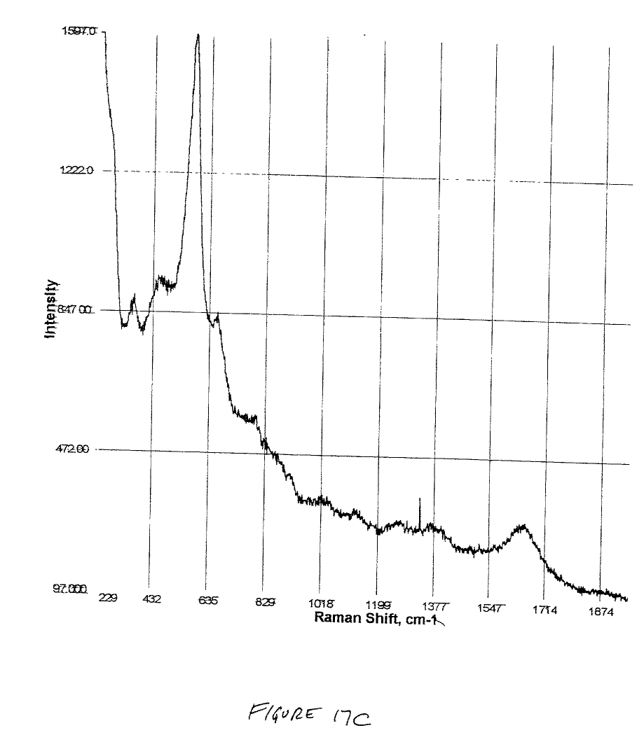

In another series of studies, the dye rhodamine was used to test the ability of fractal aggregates to enhance Raman spectroscopic detection. In one study, we applied a solution of rhodamine (10⁻¹⁶ M; “R6G”) to an aluminum foil surface having silver fractal enhancing structures thereon. FIG. 17a depicts the Raman spectrum obtained for this preparation. No Raman signal characteristic of rhodamine was observed.

We also obtained the Raman spectrum of a solution of R6G in the presence of water on the surface of a piece of aluminum foil not having any fractal structures thereon. FIG. 17b depicts the Raman spectrum of this preparation. As with FIG. 17a, no Raman spectrum characteristic of R6G was observed.

We also obtained the Raman spectrum of a sample of R6G on a piece of aluminum foil that had been prepared by the addition of silver fractal enhancing structures as described above. FIG. 17c shows that in the presence of enhancing structures, a sharp peak at about 550 cm⁻¹ was observed. This Raman spectral feature is characteristic of rhodamine.

This study indicates that using the fractal derivatized devices and methods of this invention, a molecule can be detected in concentrations as low as 10⁻¹⁶ M.

### EXAMPLE 19

### Antigen as Receptor for Detection of Antibodies by Raman Spectroscopy

To detect specific antibodies raised against 2-4 di-nitrophenol (2,4-DNP; “DNP”), substrates derivatized with silver or gold enhancing structures are prepared according to methods described above. The substrate has receptors comprising Cys-N-DNP attached to the substrate using the HS group of Cysteine, so that the DNP moiety of the receptor extends above the surface of the substrate. The substrate with receptor is then washed with NaCl to remove non-bound materials. Monoclonal or polyclonal antibodies raised against DNP are then applied to the substrate and are permitted to become associated with the DNP. The conditions of this antibody-antigen binding are known in the art and need not be described herein. After a period of time has elapsed to permit binding of anti-DNP antibody to the receptor, the substrate is washed with triple distilled, deionized water to remove unbound antibody.

Raman spectra are obtained at each step of this procedure. In the absence of receptor, the substrate having enhancing structures thereon show no Raman signal. Receptors associated with enhancing structures show a Raman spectrum characteristic for the Cys-N-DNP. When anti-DNP antibodies associate with the Cys-N-DNP, the Raman spectrum changes, and is identical to neither the Cys-N-DNP nor the anti-DNP antibody alone. Control experiments in which the receptor is cysteine or glutathione do not produce a Raman signal characteristic of receptor anti-DNP antibody association. Additional controls include the use of NH₃—CH₂—CH₂—SH. Anti-DNP antibodies do not specifically bind to NH₃—CH₂—CH₂—SH and thus, the Raman signal generated by NH₃—CH₂—CH₂—SH will be unaltered in the presence of an anti-DNP antibody. Thus these strategies provide methods for detecting specific antigen-antibody interactions by Raman spectroscopy.

### INDUSTRIAL APPLICABILITY

The devices and methods of this invention can be used in the fields of chemistry and biotechnology for the detection of analytes in complex solutions containing many different species of molecules. Additionally, the methods of this invention can be used for the detection and quantification of analytes using spectroscopic methods, including Raman spectroscopy, fluorescence spectroscopy, immunobiology and mass spectroscopy.

We claim:

1. A device for detecting spectroscopic signals, comprising:
a substrate having a metal layer thereon;
fractal aggregates on said metal layer; and
an analyte receptor near said fractal aggregates.

2. A device for detecting Raman spectroscopic signals, comprising:
a substrate having a metal layer thereon;
fractal aggregates on said metal layer; and
an analyte receptor near said fractal aggregates.

3. A device for detecting Raman spectroscopic signals, comprising:
a metal surface;
fractal aggregates on said metal surface; and
an analyte receptor near said fractal aggregates.

4. The device of claim 1, wherein said analyte receptor is sufficiently near said fractal aggregates to modify a Raman spectroscopic signal generated by an analyte that is associated with said analyte receptor.

5. A device for detecting spectroscopic signals, comprising:
a substrate having a metal layer thereon;
fractal aggregates on said metal layer; and
an analyte receptor near said fractal aggregates.

6. The device of claim 2, wherein said substrate is glass.

7. The device of claim 2, wherein said substrate is quartz.

8. The device of claim 2, wherein said metal layer is gold.

9. The device of claim 2, wherein said metal layer is aluminum.

10. The device of claim 2, wherein said fractal aggregates comprise gold.

11. The device of claim 2, wherein said fractal aggregates comprise silver.

12. The device of claim 5, wherein said analyte receptor comprises reduced glutathione.

13. The device of claim 5, wherein said analyte comprises glutathione S-transferase.

14. The device of claim 5, wherein said analyte receptor comprises an antigen.

15. The device of claim 14, wherein said analyte comprises an antibody against said antigen.

16. The device of claim 14, wherein said antigen comprises DNP.

17. The device of claim 15, wherein said antibody is a DNP antibody.

18. A biochip comprising:
a substrate having a metal layer thereon, said metal layer having at least one defined area thereon;
a plurality of particle structures associated with said at least one defined area;
said defined area having a plurality of analyte receptors preferentially localized near resonance domains of said particle structures.

19. The biochip of claim 18, wherein said particle structure is a fractal structure.

20. A biochip comprising:
a substrate having a metal layer thereon;
a plurality of particle structures having a plurality of resonance domains on said metal layer, and
a plurality of analyte receptors preferentially localized near said resonance domains, wherein said particle structures modify a signal generated by an analyte associated with said analyte receptor.

21. The biochip of claim 20, wherein said substrate is selected from the group consisting of silicon, silicon dioxide, glass, and plastics.

22. The biochip of claim 20, wherein said analyte receptors comprise reduced glutathione.

23. The biochip of claim 20, wherein said analyte receptors comprise an antigen.

24. The biochip of claim 22, wherein said analyte comprises glutathione S-transferase.

25. The biochip of claim 23, wherein said analyte comprises an antibody directed against said antigen.

26. A method for manufacturing a device for detecting spectrographic signals, comprising the steps of:
forming a substrate having a metal layer thereon;
preparing a colloidal solution of metal particles;
placing said colloidal solution on said metal layer; and
permitting said metal particles to adhere to said metal layer.

27. The method of claim 26, wherein said metal layer comprises gold.

28. The method of claim 26, wherein said colloidal solution comprises gold particles.

29. The method of claim 26, wherein said colloidal solution comprises silver particles.

30. A biochip comprising:
a substrate having a metal layer thereon;
a plurality of particle structures having a plurality of resonance domains, said plurality of particle structures on said metal layer, and
a plurality of analyte receptors preferentially localized near said resonance domains, wherein said particle structures amplify a signal generated by an analyte associated with said analyte receptor.

31. The biochip of claim 30, further comprising means for identifying each of said different areas.

32. The biochip of claim 30, wherein said defined area adapted to be observed at least in part by a detector.

33. A system for analyte detection, comprising:
a substrate having a plurality of defined areas thereon, each of said areas having:
a plurality of fractal structures having a plurality of resonance domains, and
a plurality of analyte receptors localized sufficiently near said resonance domains to enhance an electromagnetic signal generated by said analyte;
identifiers for each of said different areas; and
a detector associated with a defined area on said substrate.

34. The system of claim 33, wherein said analyte receptors comprise an antigen.

35. A method for analyte detection, comprising:
providing a substrate having a metal layer thereon;
providing a fractal aggregate on said metal layer, said fractal aggregate having at least one resonance domain;
providing an analyte receptor near said resonance domain;
exposing said analyte to said receptor; and
detecting a Raman spectral feature associated with said analyte receptor complex.

36. The method of claim 35, further comprising the step of:
removing unbound analytes from said substrate.

37. The method of claim 36, further comprising the steps of:
analyzing said spectral feature; and
comparing said spectral feature with a known reference spectral feature.

38. The method of claim 37, further comprising the step of providing an output relating to said spectral feature.

39. A method for manufacturing a device for detecting the presence of an analyte, said method comprising the steps of:
providing a substrate;
providing a layer of metal on the surface of said substrate;
providing a fractal aggregate on said layer of metal, said fractal aggregate being able to enhance an electromagnetic signal generated by said analyte; and
providing an analyte receptor on said device, sufficiently near to said fractal aggregate to enhance an electromagnetic signal generated by said analyte.

40. The method of claim 39, wherein said layer of metal is gold.

41. The method of claim 39, wherein said fractal aggregate is a silver fractal aggregate.

42. The method of claim 41, wherein said silver fractal aggregate is made comprising the steps of:
providing a colloidal solution of silver particles, comprising the steps of:
providing a solution of AgNO₃;
boiling said solution;
adding a solution of 1% sodium citrate to produce said silver particles; and
adding NaCl to said colloidal solution to produce said fractal aggregate.

43. The method of claim 41, wherein said silver fractal aggregate is made comprising the steps of:
providing a colloidal solution of silver particles, comprising the steps of:
providing a solution of NaBH₄;
Adding to said solution of NaBH₄ a solution of AgNO₃ in water while stirring;
adding a solution of poly(vinyl alcohol);
boiling said solution to form a silver colloid solution; and mixing said silver colloid solution with a solution of NaCl in water; and permitting said silver fractal aggregate to form.

44. The method of claim 39, wherein said fractal aggregate is a gold fractal aggregate.

45. The method of claim 44, wherein said fractal aggregate is made comprising the steps of:
providing a colloidal solution of gold, comprising the steps of:
providing a solution of HAuCl₄ in water;
boiling said solution; and
adding a solution of 1% sodium citrate to form said colloidal gold solution; and
mixing said colloidal gold solution with a solution of NaCl to form said gold fractal aggregates.

46. A method for manufacturing a biochip comprising the steps of:
providing a substrate having a metal surface with at least one defined area thereon;
providing a plurality of particle structures having a plurality of resonance domains;
said defined area having a plurality of analyte receptors preferentially localized near said resonance domains.

47. The method of claim 46, wherein said particle structure is a fractal structure.

48. The method of claim 46, wherein said analyte receptors comprise an antigen.

49. The method of claim 48, wherein said antigen comprises DNP.

50. The method of claim 46, wherein said analyte receptor is selected from the group consisting of Gly-Cys, Cys-Glu and reduced glutathione.

51. The method of claim 50, wherein said analyte receptor comprise reduced glutathione.

52. The method of claim 50, wherein said analyte is glutathione S-transferase.

53. A method for manufacturing a biochip comprising the steps of:
providing a substrate having a metal layer thereon;
providing a plurality of particle structures having a plurality of resonance domains on said substrate, and
providing a plurality of analyte receptors localized sufficiently near said resonance domains, wherein said particle structures enhance a signal generated by an analyte associated with said analyte receptor.

54. The method of claim 53, wherein said substrate is selected from the group consisting of silicon, silicon dioxide, glass, quartz and plastics.

55. The method of claim 53, wherein said analyte receptor is selected from the group consisting of reduced glutathione, Cys-Glu and Gly-Cys.

56. The method of claim 53, wherein said analyte receptor is an antigen.

57. The method of claim 53, wherein said analyte receptor is an antibody directed against said antigen.

58. The method of claim 56, wherein said antigen is DNP.

59. The method of claim 57, wherein said analyte is an anti-DNP antibody.

60. A method for determining the enhancing ability of a fractal aggregate, comprising the steps of:
providing a solution of fractal aggregates;
measuring a Raman signal from said solution;
adding a Raman signal generator to said solution of fractal aggregates; and
measuring a Raman signal from said Raman signal generator.

61. The method of claim 60, wherein said Raman signal generator is selected from the group consisting of DTP and rhodamine.

62. The method of claim 60, wherein said fractal aggregates are associated with a surface.

63. A device for detecting spectroscopic signals, comprising:
a substrate having a metal layer thereon;
means for enhancing an electromagnetic signal associated with said metal layer; and
an analyte receptor near said fractal aggregates.

64. A method for analyte detection, comprising:
providing a substrate having a metal layer thereon;
providing a means for enhancing an electromagnetic signal generated by said analyte;
providing means for localizing an analyte near said means for enhancing an electronic signal;
exposing said analyte to an electromagnetic beam; and
detecting a Raman spectral feature associated with said analyte receptor complex.
